(19) United States
(12) Patent Application Publication
(10) Pub. No.: US 2025/0354214 A1
(43) Pub. Date: Nov. 20, 2025

(54) MICRORNA DIAGNOSTIC ASSAY FOR CHRONIC KIDNEY DISEASE

(71) Applicant: MI:RNA LTD., EDINBURGH (GB)

(72) Inventors: Eve HANKS, EDINBURGH (GB); Robert COULTOUS, EDINBURGH (GB)

(73) Assignee: MI:RNA LTD., EDINBURGH (GB)

(21) Appl. No.: 19/210,873

(22) Filed: May 16, 2025

Related U.S. Application Data
(60) Provisional application No. 63/648,561, filed on May 16, 2024.
(60) Provisional application No. 63/767,757, filed on Mar. 6, 2025.
(60) Provisional application No. 63/775,757, filed on Mar. 21, 2025.

(51) Int. Cl. C12Q 1/6883

(52) U.S. Cl. CPC C12Q 1/6883; C12Q 2600/158; C12Q 2600/178

(57) ABSTRACT

A method for differentially assessing and diagnosing a diseased state of chronic kidney disease (CKD) in a subject, comprising the steps of: obtaining a sample from the subject; determining a level of expression of each of a plurality of miRNA molecules within the sample; comparing the level of expression of each miRNA molecule with at least one pre-determined reference level characteristic of a non-diseased subject for each one of the plurality of the miRNA molecules of step, where a deviation of the level of expression of miRNA molecules in comparison with the at least one reference level allows for the diagnosis and/or prognosis of CKD.

### CROSS REFERENCE TO RELATED APPLICATIONS

This application claims benefit of and priority to U.S. Provisional Application No. 63/648,561, filed on May 16, 2024, U.S. Provisional Application No. 63/767,757, filed on Mar. 6, 2025, and U.S. Provisional Application No. 63/775,757, filed on Mar. 21, 2025, which are hereby incorporated by reference in their entirety.

### REFERENCE TO ELECTRONIC SEQUENCE LISTING

The application contains a Sequence Listing which has been submitted electronically in .XML format and is hereby incorporated by reference in its entirety. Said .XML copy, created on Jun. 26, 2025, is named “068075.008US.xml” and is 85,865 bytes in size. The sequence listing contained in this .XML file is part of the specification and is hereby incorporated by reference herein in its entirety.

### FIELD

This invention relates generally to isolated nucleic acid molecules known as microRNAs (miRNAs) and miRNA precursor molecules and their use in diagnosis and therapy. The invention also relates to a method and a kit for diagnosing chronic kidney disease (CKD).

### BACKGROUND

Chronic kidney disease (CKD) is described as the functional or structural abnormality of one or both kidneys that has been present for an extended period of three months or more (Polzin, D. J. Vet. Clin. North Am. Small Anim, 41: 15-30 (2011)). A recent extensive UK study that merged the clinical databases of over 100,000 dogs attending 89 UK veterinary practices identified a CKD true prevalence of up to 0.37% within the general population (O'Neill, D., et al., J Vet Intern Med, 27: 814-821 (2013)), although other studies have noted a prevalence as high as 4% in university clinic populations (Sosnar, M., et al., Acta Vet. Brno, 72: 593-598 (2003)). In the feline population, A study of over 140,000 cats attending 91 UK veterinary practices noted a CKD prevalence of 3.6% (D. G. O'Neill, et al., Vet J, 202: 286-291 (2014)), with other authors noting this rises to over 30% in older feline populations >10 years of age (Jepson, R., et al., J Vet Intern Med, 23: 806-813 (2009)).

CKD is a progressive disease, with most therapies aiming to slow the loss of renal function (Polzin, D. J., 2011). Specialized nutrition is the primary therapy for renal support, in addition to resolving the effects of any deleterious co-morbidities, such as hyperphosphatemia or hypertension (Polzin, D. J., 2011; Bartges, J. W., Vet. Clin. North Am. Small Anim, 42:669-692 (2012)). Ultimately, the literature is unanimous that early intervention and management of renal disease is likely to reduce the rate of disease progression and improve the quality and quality of life (Polzin, D. J., 2011; O'Neill, D., 2013; Jepson, R., 2009; Bartges, J. W., 2012).

In practice, CKD is typically identified and staged on a progressive 1-4 scale following the guidelines of the International Renal Interest Society (IRIS) (IRIS Staging of CKD (2019); IRIS Pocket Guide to CKD (2019)). These guidelines put primary focus on the identification of elevated blood creatinine and symmetric dimethylarginine (SDMA) levels, with further sub-staging based on proteinuria and blood pressure monitoring (IRIS Staging of CKD (2019); IRIS Pocket Guide to CKD (2019)).

SDMA has been commercially available as a marker for CKD since 2015, and is available exclusively through IDEXX Laboratories (Sargent, H. J., et al., J Small Anim Pract, 62: 71-81 (2021)). It is thought to be more sensitive than serum creatinine concentration in the detection of early renal disease, however there is some evidence of elevation in greyhounds (Liffman, R, et al., Vet Clin Pathol. 47: 458-463 (2018)) and juvenile animals (Sargent, H. J., 2021), and in those with concurrent neoplasia and nephrolithiasis (Hall J A, et al., PLOS ONE 12(4): e0174854 (2017)). It has also been documented that SDMA does not perform well in cats with hyperthyroidism (Sargent, H. J., 2021) or diabetes mellitus (Langhorn, R., et al., J Vet Intern Med, 32: 57-63 (2018)). Additionally, SDMA levels may be temporarily increased by pre-renal factors and dehydration (Sargent, H. J., 2021).

The research and interest in the use of microRNA as viable markers for the early diagnosis of CKD in human medicine has gained traction over recent years (Franczyk, B., et al., Int Urol Nephrol 54, 575-588 (2022); Jing, L., et al., Front Med, 8, (2022); Sun, I. O., Lerman, L. O. Am J Physiol Renal Physiol.; 316(5):F785-F793 (2019)). The potential value of microRNA markers in for use in veterinary CKD diagnosis is rapidly being recognized, with the literature not only noting the identification of potential microRNA markers in serum and tissue (Ichii, O., et al., Res Vet Sci, 96: 299-303(2014); Grimes, J. A., et al., Am J Vet Res, 83: 426-433 (2022)), but also in urine samples (Osamu, I., et al., Urinary Exosome-Derived microRNAs Reflecting the Changes in Renal Function in Cats. Front Vet Sci, 5, (2018); Jessen, L. R., et al., J Vet Intern Med, 34: 166-175 (2020); Gòdia, M., et al., PLoS One; 17(7):e0270067 (2022); Ichii, O., et al., Front Vet Sci.; 5:289 (2018)), and for the detection of some hereditary kidney disease (Chu, C. P., et al., Sci Rep 11, 17437(2021)).

The limitations of SDMA and serum creatinine detection in certain disease scenarios, in addition to the presence of microRNA markers in urine as an alternative sample fluid, make the emerging technology of these microRNA biomarkers ideal for the early detection of disease and implementation of renal therapy to extend and improve the quality of life for CKD patients.

Therefore, disclosed herein are compositions and methods for assessment and diagnosis of CKD in a subject by analysing the expression pattern of miRNAs markers by predictive classification algorithms using a miRNA panel to discriminate CKD patients from healthy controls; assessment of the same method to discriminate clinical stage (1-4) of CKD patients.

### SUMMARY

In accordance with the purpose(s) of this invention, as embodied and broadly described herein, this invention, in one aspect, relates to A method for differentially assessing and diagnosing a diseased state of chronic kidney disease (CKD) in a subject, comprising the steps of: obtaining a sample from the subject; isolating miRNA molecules within a sample from a subject; amplifying the cDNA molecules to a detectable concentration; probing for the cDNA molecules complimentary to the desired miRNA markers; determining a level of expression of the miRNA molecules within a sample from a subject by the level of cDNA molecules probed for the desired miRNA markers; and using one or more Artificial Intelligence (AI) model to predict the disease condition of the subject; wherein the one or more AI model compares the level of expression of each cDNA molecule with at least one pre-determined reference level cDNA molecule characteristic of a non-diseased subject wherein a deviation of the level of expression of said cDNA molecule in comparison with the at least one reference level cDNA molecule allows for the diagnosis and/or prognosis of CKD. The cDNA molecule may also be a reverse compliment cDNA.

In one embodiment, the miRNA molecules comprise a panel of reference miRNAs having at least one miRNA selected from a group consisting of nucleic acid sequence having at least 95%, 97%, 98% or 99% sequence identity to SEQ ID NO: 1, 2, 3, 4, 5, 6, 7, 8, 9, 10, 11, 12, 13, 14, 15, 16, 17, 18, 19, 20, 21, 22, 23, 24, 25, 26, 26, 28, 29, 30, 31, 32, 33, 34, 35, 36, 37, 38, 39, 40, or combinations thereof, the miRNA molecules having a reverse compliment cDNA with at least 99% sequence identity to SEQ ID NO: 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or combinations thereof. In a particular embodiment, the at least one miRNA is mir144 having at least 99% sequence identity to SEQ ID NO:10, the miRNA molecules having a reverse compliment cDNA with at least 99% sequence identity to SEQ ID NO: 59.

In another embodiment, the miRNA molecules comprise a panel of reference miRNAs having at least five miRNAs selected from a group consisting of nucleic acid sequence having at least 95%, 97%, 98% or 99% sequence identity to SEQ ID NO: 1, 2, 3, 4, 5, 6, 7, 8, 9, 10, 11, 12, 13, 14, 15, 16, 17, 18, 19, 20, 21, 22, 23, 24, 25, 26, 26, 28, 29, 30, 31, 32, 33, 34, 35, 36, 37, 38, 39, 40, or combinations thereof, the miRNA molecules having a reverse compliment cDNA with at least 99% sequence identity to SEQ ID NO: 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or combinations thereof. In yet another particular embodiment, the at least five miRNAs are mir144, mir16, mir223, mir28, mir486 having at least 99% sequence identity to SEQ ID NO:10, 14, 24, 49, and 39, respectively, the miRNA molecules having a reverse compliment cDNA with at least 99% sequence identity to SEQ ID NO: 59, 63, 73, 89, and 88, respectively.

In yet another embodiment, the miRNA molecules comprise a panel of reference miRNAs having at least nine miRNAs selected from a group consisting of nucleic acid sequence having at least 95%, 97%, 98% or 99% sequence identity to SEQ ID NO: 1, 2, 3, 4, 5, 6, 7, 8, 9, 10, 11, 12, 13, 14, 15, 16, 17, 18, 19, 20, 21, 22, 23, 24, 25, 26, 26, 28, 29, 30, 31, 32, 33, 34, 35, 36, 37, 38, 39, 40, or combinations thereof, the miRNA molecules having a reverse compliment cDNA with at least 99% sequence identity to SEQ ID NO: 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or combinations thereof. In another particular embodiment, the at least nine miRNAs are let7b, mir26a, mir214, mir143, mir144, mir16, mir223, mir28, mir486 having at least 99% sequence identity to SEQ ID NO:1, 26, 21, 9, 10, 14, 24, 49, and 39, respectively, the miRNA molecules having a reverse compliment cDNA with at least 99% sequence identity to SEQ ID NO: 50, 75, 70, 58, 59, 63, 73, 89, and 88, respectively.

In another embodiment, the method further comprises the use of at least one normalizer and/or control miRNA molecule, selected from a group consisting of nucleic acid sequence having at least 95%, 97%, 98% or 99% sequence identity to SEQ ID NO: 41, 42, 43, 44, 45, 46, 47, 48, and 49, the miRNA molecules having a reverse compliment cDNA with at least 99% sequence identity to SEQ ID NO: 90, 91, 92, 93, 94, 95, 96, and 97. The normalizer or control miRNA molecule may be an off-species control miRNA molecule.

In additional embodiment, the method further comprises the step of using a machine learning algorithm for predictive modelling, In further embodiment, the method comprises the use of a combination of AI models.

In one embodiment, the subject is a mammal. In other embodiment, the subject is a dog or cat. In yet another embodiment, the sample is a biofluid selected from the group consisting of blood, urine, milk, tissue fluid, saliva, cerebrospinal fluid (CSF), feces or another biofluid. The extracted miRNAs are cell free miRNAs.

Another aspect of the invention provides kit for use in performing the method of claim 1 comprising means for determining the level of expression of miRNA molecules selected from a miRNA panel having at least nine miRNA molecules having at least 95%, 97%, 98% or 99% sequence identity to SEQ ID NO: 1, 2, 3, 4, 5, 6, 7, 8, 9, 10, 11, 12, 13, 14, 15, 16, 17, 18, 19, 20, 21, 22, 23, 24, 25, 26, 26, 28, 29, 30, 31, 32, 33, 34, 35, 36, 37, 38, 39, 40, or combinations thereof, the miRNA molecules having a reverse compliment cDNA with at least 99% sequence identity to SEQ ID NO: 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or combinations thereof.

In one embodiment the at least nine miRNA molecules are let7b, mir26a, mir214, mir143, mir144, mir16, mir223, mir28, mir486 having at least 99% sequence identity to SEQ ID NO: 1, 26, 21, 9, 10, 14, 24, 49, and 39, respectively, the miRNA molecules having a reverse compliment cDNA with at least 99% sequence identity to SEQ ID NO: 50, 75, 70, 58, 59, 63, 73, 89, and 88, respectively.

In another embodiment, the kit has a miRNA panel having at least five miRNA molecules having at least 95%, 97%, 98% or 99% sequence identity to SEQ ID NO: 1, 2, 3, 4, 5, 6, 7, 8, 9, 10, 11, 12, 13, 14, 15, 16, 17, 18, 19, 20, 21, 22, 23, 24, 25, 26, 26, 28, 29, 30, 31, 32, 33, 34, 35, 36, 37, 38, 39, 40, or combinations thereof, the miRNA molecules having a reverse compliment cDNA with at least 99% sequence identity to SEQ ID NO: 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or combinations thereof.

In one embodiment the at least five miRNAs are mir144, mir16, mir223, mir28, mir486 having at least 99% sequence identity to SEQ ID NO:10, 14, 24, 49, and 39, respectively, the miRNA molecules having a reverse compliment cDNA with at least 99% sequence identity to SEQ ID NO: 59, 63, 73, 89, and 88, respectively.

In another embodiment, the kit has a miRNA panel having at least 1 miRNA, mir144, having at least 99% sequence identity SEQ ID NO:10, the miRNA molecule having a reverse compliment cDNA with at least 99% sequence identity to SEQ ID NO: 59.

One other aspect of the invention provides a method of selecting a panel for use in disease diagnosis comprising the steps of: selecting a group of miRNA molecules the differential expression of which may be associated with a disease condition; predicting the disease condition based on a deviation of the level of expression of said miRNA molecules from step (a) and (b); and reducing the number of miRNAs in the panel to a minimum number to provide a panel of miRNAs that still produces a result; wherein the disease is CKD.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

FIG. 1 shows the latent Dirichlet allocation (LDA) model statistics for Canine CKD versus control samples.

FIG. 2 is a principal component analysis (PCA) biplot of processed miRNA profiles that distinguish canine healthy controls from canine CKD cases using RT-qPCR data.

FIG. 3 is a microRNA expression profile heatmap of healthy controls and CKD cases. Heatmap representation of 5 miRNA expression profiles in 59 samples divided into two groups according to their disease status, healthy controls (right side; top row) or CKD cases (left side; in top row). MicroRNA expression is represented as a color gradient from low to high (blue to red) based on normalized expression.

FIG. 4 is a ten-fold cross-validated receiver operating curve (ROC) for healthy controls vs. CKD cases is shown. Cross validation was repeated five times. Area under the curve (AUC) is shown.

FIG. 5 is a principal component analysis (PCA) biplot of the analysis of feline CKD RT-qPCR data.

FIG. 6 shows the trained Gaussian process model statistics for Feline CKD versus control samples.

FIG. 7 is a heat map of the analysis of feline CKD RT-qPCR data.

FIGS. 8A-8E show the accuracy metrics of various machine learning-trained models.

FIG. 9 shows violin plots showing the distribution of normalized expression for each miRNA in CKD positive and control samples.

### DETAILED DESCRIPTION

The present invention may be understood more readily by reference to the following detailed description of preferred embodiments of the invention and the Examples included therein and to the Figures and their previous and following description.

### I. Definitions

To facilitate an understanding of the principles and features of the various embodiments of the disclosure, various illustrative embodiments are explained herein. Although exemplary embodiments of the disclosure are explained in detail, it is to be understood that other embodiments are contemplated. Accordingly, it is not intended that the disclosure is limited in its scope to the details of construction and arrangement of components set forth in the description or examples. The disclosure is capable of other embodiments and of being practiced or carried out in various ways.

Unless defined otherwise, all technical and scientific terms used herein have the same meaning as commonly understood by one of ordinary skill in the art to which the invention pertains. Although other methods and materials similar, or equivalent, to those described herein can be useful in the present invention, preferred materials and methods are described herein.

Unless otherwise specified, the experimental methods, detection methods, and preparation methods disclosed in the present invention all adopt the conventional molecular biology, biochemistry, microbiology, cell biology, genomics, and recombinant polynucleotides, chromatin structure and analysis, analytical chemistry, cell culture, recombinant DNA technology and related fields in the technical field. These techniques have been well described in the existing literature. For details, please refer to inter alia Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolfe, CHROMATIN STRUCTURE AND FUNCTION, Third edition, Academic Press, San Diego, 1998; METHODS IN ENZYMOLOGY, Vol. 304, Chromatin (P M Wassarman and A P Wolffe, eds.), Academic Press, San Diego, 1999; and METHODS IN MOLECULAR BIOLOGY, Vol. 119, Chromatin Protocols (P B Becker, ed.) Humana Press, Totowa, 1999, et al.; Cellular and Molecular Immunology, Ninth Edition, A. K. Abbas, et al., Elsevier (2017), ISBN 978-0323479783; Cancer Immunotherapy Principles and Practice, First Edition, L. H. Butterfield, et al., Demos Medical (2017), ISBN 978-1620700976; Janeway's Immunobiology, Ninth Edition, Kenneth Murphy, Garland Science (2016), ISBN 978-0815345053; Clinical Immunology and Serology: A Laboratory Perspective, Fourth Edition, C. Dorresteyn Stevens, et al., F. A. Davis Company (2016), ISBN 978-0803644663; Antibodies: A Laboratory Manual, Second edition, E. A. Greenfield, Cold Spring Harbor Laboratory Press (2014), ISBN 978-1-936113-81-1; Culture of Animal Cells: A Manual of Basic Technique and Specialized Applications, Seventh Edition, R. I. Freshney, Wiley-Blackwell (2016), ISBN 978-1118873656; Transgenic Animal Technology, Third Edition: A Laboratory Handbook, C. A. Pinkert, Elsevier (2014), ISBN 978-0124104907; The Laboratory Mouse, Second Edition, H. Hedrich, Academic Press (2012), ISBN 978-0123820082; Manipulating the Mouse Embryo: A Laboratory Manual, Fourth Edition, R. Behringer, et al., Cold Spring Harbor Laboratory Press (2013), ISBN 978-1936113019; PCR 2: A Practical Approach, M. J. McPherson, et al., IRL Press (1995), ISBN 978-0199634248; Methods in Molecular Biology (Series), J. M. Walker, ISSN 1064-3745, Humana Press; RNA: A Laboratory Manual, D. C. Rio, et al., Cold Spring Harbor Laboratory Press (2010), ISBN 978-0879698911; Methods in Enzymology (Series), Academic Press; Molecular Cloning: A Laboratory Manual (Fourth Edition), M. R. Green, et al., Cold Spring Harbor Laboratory Press (2012), ISBN 978-1605500560; Bioconjugate Techniques, Third Edition, G. T. Hermanson, Academic Press (2013), ISBN 978-0123822390; Methods in Plant Biochemistry and Molecular Biology, W. V. Dashek, CRC Press (1997), ISBN 978-0849394805; Plant Cell Culture Protocols (Methods in Molecular Biology), V. M. Loyola-Vargas, et al., Humana Press (2012), ISBN 978-1617798177; Plant Transformation Technologies, C. N. Stewart, et al., Wiley-Blackwell (2011), ISBN 978-0813821955; Recombinant Proteins from Plants (Methods in Biotechnology), C. Cunningham, et al., Humana Press (2010), ISBN 978-1617370212; Plant Genomics: Methods and Protocols (Methods in Molecular Biology), W. Busch, Humana Press (2017), ISBN 978-1493970018; Plant Biotechnology: Methods in Tissue Culture and Gene Transfer, R. Keshavachandran, et al., Orient Blackswan (2008), ISBN 978-8173716164.

In describing the exemplary embodiments, specific terminology will be resorted to for the sake of clarity. As used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named. Thus, for example, reference to “a polynucleotide” includes one or more polynucleotides, and reference to “a vector” includes one or more vectors.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value.

Similarly, as used herein, “substantially free” of something, or “substantially pure”, and like characterizations, can include both being “at least substantially free” of something, or “at least substantially pure”, and being “completely free” of something, or “completely pure.”

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

The phrase “nucleic acid” or “polynucleotide sequence” refers to a single or double-stranded polymer of deoxyribonucleotide or ribonucleotide bases read from the 5′ to the 3′ end. Nucleic acids may also include modified nucleotides that permit correct read-through by a polymerase and do not alter expression of a polypeptide encoded by that nucleic acid.

A “coding sequence” or “coding region” refers to a nucleic acid molecule having sequence information necessary to produce a gene product when the sequence is expressed.

A “probe” is defined as a nucleic acid capable of binding to a target nucleic acid of complementary sequence through one or more types of chemical bonds, usually through complementary base pairing, usually through hydrogen bond formation. A probe may include natural (i.e., A, G, C, T or U) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. Probes may bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

As used herein, the term “microRNA” or “miRNA” or “miR” designates a non-coding RNA molecule having a length of about 17 to 25 nucleotides, specifically having a length of 17, 18, 19, 20, 21, 22, 23, 24 or 25 nucleotides which hybridizes to and regulates the expression of a coding messenger RNA.

The term “miRNA molecule” refers to any nucleic acid molecule representing the miRNA, including natural miRNA molecules, i.e. the mature miRNA, pre-miRNA, pri-miRNA.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high-performance liquid chromatography. A protein that is the predominant species present in a preparation is substantially purified. In particular, an isolated nucleic acid of the present invention is separated from open reading frames that flank the desired gene and encode proteins other than the desired protein. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The term “sample” generally refers to tissue or organ sample, blood, cell-free blood such as serum and plasma, urine, saliva, milk, and cerebrospinal fluid sample.

As used herein, the term “blood sample” refers to serum, plasma, cell-free blood, whole blood and its components, blood derived products or preparations. Plasma and serum are very useful as shown in the examples.

The term “quantifying” or “quantification” as used herein refers to absolute quantification, i.e. determining the amount of the respective miRNA but also encompasses measuring the level of the respective miRNA and comparing said level with reference or control miRNA, or comparative expression to other quantified miRNAs. Quantification of the respective miRNA as listed in the tables herein allow expression profiling of samples and thus allow identification of signatures associated with diseased samples, as well as identification of signatures associated with prognosis and response to treatment. The quantity of miRNAs or difference in miRNA levels can be determined by any of the methods described herein.

A “control”, “control sample”, or “reference value” or “reference level” are terms which can be used interchangeably herein, and are to be understood as a sample or standard used for comparison with the experimental sample. The control may include a sample obtained from a healthy or non-diseased subject or a subject, which is not at risk of or suffering from CKD. Reference level specifically refers to the level of miRNA or miRNA expression quantified in a sample from a healthy subject, from a subject, which is not at risk of or suffering from CKD. Specifically, a more than 1.3, 1.4, 1.5, 1.6, 1.7, 1.8, 1.9 or 2.0 fold difference between the reference level of one or more miRNAs as defined herein obtained from a sample of a subject. Additionally, a control may also be a standard reference value or range of values, i.e. such as stable expressed miRNAs in the samples, for example the endogenous control.

“Animal(s)”, as used herein, unless otherwise indicated, refers to an individual animal that is a mammal. Specifically, mammal refers to a vertebrate animal that is human and non-human, which are members of the taxonomic class Mammalia. Non-exclusive examples of non-human mammals include companion animals. Non-exclusive examples of a companion animal include: dog, cat, and horse.

As used herein, the terms “wild-type,” “naturally occurring,” and “unmodified” refer to the typical (or most common) form, appearance, phenotype, or strain existing in nature; for example, the typical form of cells, organisms, polynucleotides, proteins, macromolecular complexes, genes, RNAs, DNAs, or genomes as they occur in, and can be isolated from, a source in nature. The wild-type form, appearance, phenotype, or strain serve as the original parent before an intentional modification. Thus, mutant, variant, engineered, recombinant, and modified forms are not wild-type forms.

The terms “oligonucleotide” and “polynucleotide” as used interchangeably herein refer to a polymer of greater than one nucleotide in length of ribonucleic acid (RNA), deoxyribonucleic acid (DNA), hybrid RNA/DNA, modified RNA or DNA, or RNA or DNA mimetics. The polynucleotides may be single- or double-stranded. The terms include polynucleotides composed of naturally-occurring nucleobases, sugars and covalent internucleoside (backbone) linkages as well as polynucleotides having non-naturally-occurring portions which function similarly. Such modified or substituted polynucleotides are well-known in the art and for the purposes of the present invention, are referred to as “analogues.”

As used herein, the term “polypeptide” refers to a chain of amino acids of any length, regardless of modification (e.g., phosphorylation or glycosylation). The term polypeptide includes proteins and fragments thereof. The polypeptides can be “exogenous,” meaning that they are “heterologous,” i.e., foreign to the host cell being utilized, such as human polypeptide produced by a bacterial cell. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, the term “variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of the disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and cofactors. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

The term “percent (%) sequence identity” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For purposes herein, the % sequence identity of a given nucleotides or amino acids sequence C to, with, or against a given nucleic acid sequence D (which can alternatively be phrased as a given sequence C that has or comprises a certain % sequence identity to, with, or against a given sequence D) is calculated as follows:

$$100 times the fraction W / Z ,$$

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

The terms “primer” and “polynucleotide primer,” as used herein, refer to a short, single-stranded polynucleotide capable of hybridizing to a complementary sequence in a nucleic acid sample. A primer serves as an initiation point for template-dependent nucleic acid synthesis. Nucleotides are added to a primer by a nucleic acid polymerase in accordance with the sequence of the template nucleic acid strand. A “primer pair” or “primer set” refers to a set of primers including a 5′ upstream primer that hybridizes with the 5′ end of the sequence to be amplified and a 3′ downstream primer that hybridizes with the complementary 3′ end of the sequence to be amplified. The term “forward primer” as used herein, refers to a primer which anneals to the 5′ end of the sequence to be amplified. The term “reverse primer”, as used herein, refers to a primer which anneals to the complementary 3′ end of the sequence to be amplified.

The terms “probe” and “polynucleotide probe,” as used herein, refer to a polynucleotide used for detecting the presence of a specific nucleotide sequence in a sample. Probes specifically hybridize to a target nucleotide sequence, or the complementary sequence thereof, and may be single- or double-stranded.

The terms “annealing” and “hybridization” are used interchangeably and mean the base-pairing interaction of one nucleic acid with another nucleic acid that results in formation of a duplex or other higher-ordered structure. The primary interaction is base specific, i.e. A/T and G/C, by Watson/Crick and Hoogsteen-type hydrogen bonding.

As used herein, the phrase “hybridization conditions” or “stringent hybridization conditions” refers to hybridization conditions which can take place under a number of pH, salt and temperature conditions. The pH can vary from 6 to 9, preferably 6.8 to 8.5. The salt concentration can vary from 0.15 M sodium to 0.9 M sodium, and other cations can be used as long as the ionic strength is equivalent to that specified for sodium. The temperature of the hybridization reaction can vary from 30° C. to 80° C., preferably from 45° C. to 70° C. Additionally, other compounds can be added to a hybridization reaction to promote specific hybridization at lower temperatures, such as at or approaching room temperature. Among the compounds contemplated for lowering the temperature requirements is formamide. Thus, a polynucleotide is typically “substantially complementary” to a second polynucleotide if hybridization occurs between the polynucleotide and the second polynucleotide. As used herein, “hybridization” or “specific hybridization” refers to hybridization between two polynucleotides under stringent hybridization conditions.

The term “specifically hybridize,” as used herein, refers to the ability of a polynucleotide to bind detectably and specifically to a target nucleotide sequence. Polynucleotides, oligonucleotides and fragments thereof specifically hybridize to target nucleotide sequences under hybridization and wash conditions that minimize appreciable amounts of detectable binding to non-specific nucleic acids. High stringency conditions can be used to achieve specific hybridization conditions as is known in the art. Typically, hybridization and washing are performed at high stringency according to conventional hybridization procedures and employing one or more washing step in a solution comprising 1-3×SSC, 0.1-1% SDS at 50-70° C. for 5-30 minutes.

As used herein, the term “hybridizes under stringent conditions” describes conditions for hybridization and washing under which nucleotide sequences having at least 30%, 35%, 40%, 45%, 50%, 55%, 60%, 65%, 70%, 75%, 80%, 85%, 90%, 95%, or more base pair matches to each other typically remain hybridized to each other.

The term “corresponding to” refers to a polynucleotide sequence that is identical to all or a portion of a reference polynucleotide sequence. In contradistinction, the term “complementary to” is used herein to indicate that a polynucleotide sequence is identical to all or a portion of the complementary strand of a reference polynucleotide sequence.

The terms “target sequence” or “target nucleotide sequence,” as used herein, refer to a particular nucleic acid sequence in a test sample to which a primer and/or probe is intended to specifically hybridize. A “target sequence” is typically longer than the primer or probe sequence and thus can contain multiple “primer target sequences” and “probe target sequences.” A target sequence may be single or double stranded. The term “primer target sequence” as used herein refers to a nucleic acid sequence in a test sample to which a primer is intended to specifically hybridize. The term “probe target sequence” refers to a nucleic acid sequence in a test sample to which a probe is intended to specifically hybridize.

As used herein an “amplified target polynucleotide sequence product” or “amplified product” or “amplification product” refers to the resulting amplicon from an amplification reaction such as a polymerase chain reaction. The resulting amplicon product arises from hybridization of complementary primers to a target polynucleotide sequence under suitable hybridization conditions and the repeating in a cyclic manner the polymerase chain reaction as catalyzed by DNA polymerase for DNA amplification or RNA polymerase for RNA amplification.

As used herein, the “polymerase chain reaction” or PCR is a an amplification of nucleic acid consisting of an initial denaturation step which separates the strands of a double stranded nucleic acid sample, followed by repetition of (i) an annealing step, which allows amplification primers to anneal specifically to positions flanking a target sequence; (ii) an extension step which extends the primers in a 5′ to 3′ direction thereby forming an amplicon polynucleotide complementary to the target sequence, and (iii) a denaturation step which causes the separation of the amplicon from the target sequence (Mullis et al., eds, The Polymerase Chain Reaction, BirkHauser, Boston, Mass. (1994). Each of the above steps may be conducted at a different temperature, preferably using an automated thermocycler (Applied Biosystems LLC, a division of Life Technologies Corporation, Foster City, Calif). If desired, RNA samples can be converted to DNA/RNA heteroduplexes or to duplex cDNA by methods known to one of skill in the art.

As used herein, “amplifying” and “amplification” refers to a broad range of techniques for increasing polynucleotide sequences, either linearly or exponentially. Exemplary amplification techniques include, but are not limited to, PCR or any other method employing a primer extension step. Other nonlimiting examples of amplification include, but are not limited to, ligase detection reaction (LDR) and ligase chain reaction (LCR). Amplification methods may comprise thermal-cycling or may be performed isothermally. In various embodiments, the term “amplification product” or “amplified product” or “amplification product” includes products from any number of cycles of amplification reactions.

In certain embodiments, amplification methods comprise at least one cycle of amplification, for example, but not limited to, the sequential procedures of: hybridizing primers to primer-specific portions of target sequence or amplification products from any number of cycles of an amplification reaction; synthesizing a strand of nucleotides in a template-dependent manner using a polymerase; and denaturing the newly-formed nucleic acid duplex to separate the strands. The cycle may or may not be repeated.

Descriptions of certain amplification techniques can be found, among other places, in H. Ehrlich et al., Science, 252:1643-50 (1991), M. Innis et al., PCR Protocols: A Guide to Methods and Applications, Academic Press, New York, N.Y. (1990), R. Favis et al., Nature Biotechnology 18:561-64 (2000), and H. F. Rabenau et al., Infection 28:97-102 (2000); Sambrook and Russell, Molecular Cloning, Third Edition, Cold Spring Harbor Press (2000) (hereinafter “Sambrook and Russell”), Ausubel et al., Current Protocols in Molecular Biology (1993) including supplements through September 2005, John Wiley & Sons (hereinafter “Ausubel et al.”).

As used herein, the term “sample” is a portion of a larger source. A sample is optionally a solid, gaseous, or fluidic. A sample is illustratively an environmental or biological sample. An environmental sample is illustratively, but not limited to water, sewage, soil, or air. A “biological sample” is a sample obtained from a biological organism, a tissue, cell, cell culture medium, or any medium suitable for mimicking biological conditions. Non-limiting examples include urine, saliva, gingival secretions, cerebrospinal fluid, gastrointestinal fluid, mucous, urogenital secretions, synovial fluid, blood, serum, plasma, feces, cystic fluid, lymph fluid, ascites, pleural effusion, interstitial fluid, intracellular fluid, ocular fluids, seminal fluid, mammary secretions, vitreal fluid, throat, and nasal secretions, and the like. Methods of obtaining a sample are known in the art. In one embodiment of the present invention, the sample is urine and is collected by any method well known in the art including by catheterization of a feline subject and, optionally, may be processed to obtain the final sample.

As used herein, the term “medium” refers to any liquid or fluid that may or may not contain one or more bacteria. A medium is illustratively a solid sample that has been suspended, solubilized, or otherwise combined with fluid to form a fluidic sample. Non-limiting examples include buffered saline solution, cell culture medium, acetonitrile, trifluoroacetic acid, combinations thereof, or any other fluid recognized in the art as suitable for combination with bacteria or other cells, or for dilution of a biological sample or amplification product for analysis.

As used herein, the term “pharmaceutically acceptable carrier” encompasses any of the standard pharmaceutical carriers, such as a phosphate buffered saline solution, water, and emulsions such as an oil/water or water/oil emulsion, and various types of wetting agents.

As used herein, the term “fibrotic disorder” or “fibrotic disease” refers to a medical condition featuring progressive and/or irreversible fibrosis, wherein excessive deposition of extracellular matrix occurs in and around inflamed or damaged tissue.

The polymerase chain reaction (PCR) is a primer extension reaction that provides a method to amplify a specific DNA or polynucleotide in vitro, generating thousands to millions of copies of a particular DNA sequence. PCR is now a common and often indispensable technique used in medical and biological research labs for a variety of applications. These include DNA cloning for sequencing, DNA-based phylogeny, or functional analysis of genes; the diagnosis of hereditary diseases; the identification of genetic fingerprints; and the detection and diagnosis of infectious diseases. Some of the variations of the basic PCR include quantitative real-time PCR (qPCR or RT-PCR), allele specific PCR, asymmetric PCR, hot start PCR, reverse transcription PCR, multiplex-PCR, nested-PCR, ligation-mediated PCR, Intersequence-specific PCR, Thermal asymmetric interlaced PCR and touchdown-PCR. These PCR variations provide a wide variety of uses for different purposes. For example, single-nucleotide polymorphisms (SNPs) (single-base differences in DNA) can be identified by allele-specific PCR, qPCR can provide a very high degree of precision in determining the number of copies amplified in the PCR reactions (Bartlett et al., “A Short History of the Polymerase Chain Reaction”, PCR Protocols, 2003).

### II. Compositions

The present invention provides genomic identifiers for CKD. These can be used as target nucleic acid sequences for diagnosis of CKD in a subject. The diagnostic targets can be used for identification of CKD in a sample.

One aspect of the present invention is directed to compositions and methods relating to an assay for the diagnosis and staging of CKD. In one embodiment of the present invention, an assay that is rapid, reliable, and can be used for detecting the presence of a target indicator in body fluids. It is especially advantageous that an assay in accordance with the present invention can not only be useful in diagnosing CKD, but also can distinguish early-stage disease before clinical symptoms occur and during which intervention and treatment may limit progression of disease.

The practice of the present invention employs, unless otherwise indicated, conventional techniques of molecular biology (including recombinant techniques), microbiology, cell biology, biochemistry, immunology, protein kinetics, and mass spectroscopy, which are within the skill of art. Such techniques are explained fully in the literature, such as Sambrook et al., 2000, Molecular Cloning: A Laboratory Manual, third edition, Cold Spring Harbor Laboratory Press; Current Protocols in Molecular Biology Volumes 1-3, John Wiley & Sons, Inc.; Kriegler, 1990, Gene Transfer and Expression: A Laboratory Manual, Stockton Press, New York; Dieffenbach et al., 1995, PCR Primer: A Laboratory Manual, Cold Spring Harbor Laboratory Press, each of which is incorporated herein by reference in its entirety. Procedures employing commercially available assay kits and reagents typically are used according to manufacturer-defined protocols unless otherwise noted.

Generally, the nomenclature and the laboratory procedures in recombinant DNA technology described below are those well-known and commonly employed in the art. Standard techniques are used for cloning, DNA and RNA isolation, amplification, and purification. Generally enzymatic reactions involving DNA ligase, DNA polymerase, restriction endonucleases and the like are performed according to the manufacturer's specifications.

Provided herein is a method for assessing and diagnosing CKD in a subject, comprising the steps of: (a) determining the level of expression of each of a plurality of miRNAs within a sample from a subject; and (b) using one or more Artificial Intelligence (AI) model to predict the disease condition of the subject.

A. mRNAs

Nucleotide sequences of mature miRNAs and their respective precursors are known in the art and available from the database miRBase or from Sanger database.

Identical polynucleotides as used herein in the context of a polynucleotide to be detected by the method as described herein may have a nucleic acid sequence with an identity of at least 90%, 95%, 97%, 98% or 99% or less than 3 or 2 single nucleotide modifications compared to a polynucleotide comprising or consisting of a reference nucleotide sequence of any one of SEQ ID NOs:1-40.

Furthermore, identical polynucleotides as used herein in the context of a polynucleotide to be detected by the method as described herein may have a nucleic acid sequence with an identity of at least 90%, 95%, 97%, 98% or 99% to a polynucleotide comprising or consisting of the nucleotide sequence of any one of SEQ ID NOs: 1-40 including one, two, three or more nucleotides of the corresponding pre-miRNA sequence at the 5′end and/or the 3′end of the respective seed sequence.

All of the specified miRNAs used according to the invention also encompass isoforms and variants thereof. For the purpose of the invention, the terms “isoforms and variants” (which have also be termed “isomirs”) of a reference miRNA include trimming variants (5′ trimming variants in which the 5′ dicing site is upstream or downstream from the reference miRNA sequence; 3′ trimming variants: the 3′ dicing site is upstream or downstream from the reference miRNA sequence), or variants having one or more nucleotide modifications (3′ nucleotide addition to the 3′ end of the reference miRNA; nucleotide substitution by changing nucleotides from the miRNA precursor), or the complementary mature microRNA strand including its isoforms and variants (for example for a given 5′ mature microRNA the complementary 3′ mature microRNA and vice-versa). With regard to nucleotide modification, the nucleotides relevant for RNA/RNA binding, i.e. the 5′-seed region and nucleotides at the cleavage/anchor side are excluded from modification.

In the following, if not otherwise stated, the term “miRNA” encompasses 3p and 5p strands and their isoforms and variants.

The plurality of miRNAs form a panel comprising the following: let-7b, miR-10a, miR-10b, miR-126, miR-130a, miR-132, miR-135a, miR-135b, miR-143, miR-144, miR-146b, miR-150, miR-155, miR-16, miR-200a, miR-200b, miR-200c, miR-204, miR-206, miR-21, miR-214, miR-217, miR-21a, miR-223, miR-25, miR-26a, miR-27a, miR-29a, miR-29b, miR-29c, miR-30a, miR-30b, miR-30c, miR-30d, miR-30e, miR-34a, miR-382, miR-451, miR-486, and miR-802 as set out below in Table 1.

The method further comprises the use of at least one normalizer and/or an off-species control miRNA molecule. At least one normalizer is used to ‘normalize’ data, i.e. to control for variation between the samples tested in the method of the invention, and the at least one control is used to try to ensure there are no failure or false readings in the results. An off-species control is added in to show that the miRNAs detected are relevant to the species panel. The off-species control is a miRNA from another species, i.e. not a ruminant. Advantageously, the use of an off-species controls provides another layer of control to distinguish between background or non-specific signals and a positive result. The sequences of the normalizers and the off-species controls that were used are provided below in Table 1.

It is preferred that the method comprises the step of assessing the relative levels of miRNA expression of each one of miRNA molecules let-7b, miR-10a, miR-10b, miR-126, miR-130a-3p, miR-132, miR-135a, miR-135b, miR-143, miR-144, miR-146b, miR-150, miR-155, miR-16, miR-200a, miR-200b, miR-200c, miR-204, miR-206, miR-21, miR-214, miR-217, miR-21a, miR-223, miR-25, miR-26a, miR-27a, miR-29a, miR-29b, miR-29c, miR-30a, miR-30b, miR-30c, miR-30d, miR-30e, miR-34a, miR-382, miR-451, miR-486, and miR-802 within a sample from a subject and using the data obtained from measurement of the expression levels to determine the presence or absence of disease in a subject.

The plurality of target miRNAs form a panel comprising the following: let-7b, miR-10a, miR-10b, miR-126, miR-130a-3p, miR-132, miR-135a, miR-135b, miR-143, miR-144, miR-146b, miR-150, miR-155, miR-16, miR-200a, miR-200b, miR-200c, miR-204, miR-206, miR-21, miR-214, miR-217, miR-21a, miR-223, miR-25, miR-26a, miR-27a, miR-29a, miR-29b, miR-29c, miR-30a, miR-30b, miR-30c, miR-30d, miR-30e, miR-34a, miR-382, miR-451, miR-486, and miR-802.

### III. Methods of Detection

### A. Identification of CKD

Chronic kidney disease (CKD), a progressive and irreversible condition that affects the kidneys of cats and dogs.

### 1. Chronic Kidney Disease in Cats

CKD is a common disease in older cats and can lead to serious health complications if left untreated. Interestingly, when data in dogs and cats were similarly obtained, the prevalence of CKD in geriatric cats exceeded that observed in geriatric dogs by 2-fold or more. The prevalence increases in cats from 5 to 6 years onward, with estimates of CKD in geriatric cats ranging from 35% to 81%. For example, a recent retrospective study by the Royal Veterinary College (RVC) reported that about 30% of cats aged 10 years or older have CKD, which equates to approximately 600,000 cases alone in the UK. Of these, about 40% will also have hypertension. Other studies report that CKD may affect as many as 50% of elderly cats, with prevalence increasing with age (Marino, C. L. et al, J Feline Med Surg., 16(6):465-472 (2013)).

A second trend is the increasing prevalence of the diagnosis of CKD in cats during recent decades. Data from the Purdue Veterinary Medical Database suggests that the overall prevalence of feline CKD increased from 0.04% in the 1980s to 0.2% in 1990s to 1% by the 2000s (Brown, C. A., et al., Vet Pathol., 53(2):309-236 (2016)). Whether this increase is a reflection of increased awareness with enhanced diagnostic acumen, an increase in the median age within cat populations, or a true increase in prevalence is unknown.

The diagnosis of feline CKD is based on a combination of clinical signs, laboratory tests, and imaging studies. The most common clinical signs of CKD in cats include increased thirst and urination, weight loss, poor appetite, vomiting, and lethargy. Laboratory tests such as blood chemistry, urinalysis, and urine culture can help to confirm the diagnosis of CKD. Imaging studies such as ultrasound can also be used to evaluate the kidneys and assess the severity of the disease.

Feline CKD is categorized into 4 stages based on, amongst other signs, creatinine levels and pathological changes in the kidney associated with each stage have been described. Stage 1 or early kidney disease is characterized as associated with mild kidney damage and no clinical signs, although lesions that involve as little as 25%-50% of the parenchyma can impact function. Stage 2 is classified as mild to moderate kidney disease, with mild clinical signs, while Stage 3 is described as moderate to severe kidney disease and Stage 4 characterized as end-stage kidney disease with severe clinical signs. Although progressive fibrosis is considered to be a hallmark of the disease, there appears to be no significant difference in the percentage of fibrosis between stage 2 and stage 3, leading to the hypothesis that factors other than fibrosis may contribute to later stage disease progression.

Currently, treatment of CKD includes managing clinical signs and attempting to slow progression of the disease. Treatment options include providing a special diet low in protein and phosphorus to help reduce kidney workload and prescribing medications, such as ACE inhibitors, phosphate binders, and erythropoietin, to help to manage the clinical signs and improve the cat's quality of life. Fluid replacement therapy can be provided as an adjunct to other treatments to maintain hydration and improve kidney function. Regular monitoring of the cat's kidney function and clinical signs is essential to assess the effectiveness of the treatment, while complications such as anemia, hypertension, and urinary tract infections should be managed promptly to prevent further damage to the kidneys. Life expectancy of a cat with CKD depends on several factors, including the stage of the disease, the cat's age, and the presence of other medical conditions. However, in general, cats with CKD have a reduced life expectancy compared to healthy cats, but with appropriate treatment and management, many cats with CKD can live for several years.

The exact cause of feline CKD is not fully understood, but there are several factors that can contribute to the development of the disease, including age because not only is CKD more common in older cats, but the risk of developing the disease also increases with age. Genetics is also thought to play a role because certain breeds, such as Persians and Siamese cats, are more prone to developing the disease. Chronic infections of the urinary tract or kidneys can lead to kidney damage and subsequently to the development of CKD as can exposure to certain toxins, such as antifreeze, which can cause kidney damage. Other medical conditions, especially those associated with damage to kidney function such as diabetes, hyperthyroidism, and hypertension, are known to increase the risk of a cat developing CKD. However, it is important to note that in many cases the exact cause of feline CKD is unknown, and the disease may develop as a result of a combination of factors.

Fibrosis, a common feature of feline CKD, is a process in which excess connective tissue is deposited in the kidneys leading to scarring and loss of function. Thus, fibrosis and fibrosis activating factor (FAF), a protein that has been identified as a potential contributor to the development of fibrosis in the kidneys, is thought to play a significant role in the progression of the disease. FAF, produced by kidney cells in response to injury or inflammation, stimulates the production of fibroblasts which then produce connective tissue. Studies have shown that FAF levels are elevated in cats with CKD, and that higher levels of FAF are associated with more severe fibrosis and kidney damage, supporting the role FAF may have in the development and progression of fibrosis in feline CKD. Although research is ongoing to better understand the role of FAF, as well as to develop treatments that target this protein as a way to slow or prevent the progression of the disease, there currently are no such specific treatments.

There is also great interest amongst researchers and clinicians to diagnose early stage CDK because intervention then may be most useful for decreasing mortality and morbidity associated with later stage CDK. Unfortunately, current diagnosis relies upon identification of clinical signs, laboratory testing, and imaging results and none are able to quickly and accurately stage the disease until progression is quite far along. For example, IDEXX promotes their SDMA test as being able to diagnose CKD at an earlier stage than when relying on creatinine levels, when there has been 20%-40% loss of kidney function (prior to stage IV).

### 2. Chronic Kidney Disease in Dogs

Chronic kidney disease affects dogs of all ages, but more commonly older dogs with a prevalence of up to 7% in dogs (Polzin D J. Vet Clin North Am Small Anim Pract. 41:15-30(2011); Lund E M, et al.; J Am Vet Med Assoc. 214:1336-1341 (1999); O'Neill D G, et al. J Vet Intern Med.; 27:814-821 (2013)). It is a progressive disease in dogs, but progression rate is highly variable where it may be gradual and constant or a consequence of sequential acute kidney injury (AKI) episodes of variable magnitude (Polzin D J 2011; Bartges J W. Vet Clin North Am Small Anim Pract. 42:669-692 (2012); Cowgill L D, et al. Vet Clin North Am Small Anim Pract. 46:995-1013 (2016). Recognized markers associated with the progression and outcome of CKD in dogs include presence of anemia, low body condition score, proteinuria, hypertension, hypoalbuminemia, and International Renal Interest Society (IRIS) stage (Polzin D J, 2011; Bartges J W., 2012; Cowgill L D, 2016; Parker V J, Freeman L M. J Vet Intern Med. 25:1306-1311 (2011); Jacob F, et al. J Am Vet Med Assoc.; 226:393-400 (2005)). Chronic kidney disease in dogs may be familial, but it has been found to be more of an acquired condition (O'Neill D G, et al. 2013). Causes that have been implicated in the pathogenesis of CKD include glomerular diseases, infections (e.g., chronic pyelonephritis), repeated ischemic events, nephrotoxicity, neoplasia, previous AKI or urinary obstruction, but often the etiology is unknown at presentation and remains unidentified throughout the disease course (O'Neill D G, et al. 2013); Cowgill L D 2016; Rudinsky A J, et al. J Vet Intern Med. 32:1977-1982 (2018)). Unfortunately, due to the limitations of currently available diagnostic markers, CKD often is diagnosed late and at a point when renal functional impairment exceeds compensatory mechanisms, and irreversible, severe renal parenchymal damage already has occurred.

Animals with stable CKD may experience an acute decrease in kidney function (i.e., acute on chronic kidney disease (ACKD)). The pathogenesis, clinical presentation and laboratory abnormalities of ACKD may resemble those of AKI (Cowgill L D, 2016), which occasionally makes differentiation between AKI and ACKD challenging. Although ACKD is common (Rudinsky A J, 2018) its causes, clinical course and short- and long-term outcomes have yet to be described in dogs.

### 3. Stages of CKD

CKD is categorized into disease stages established by the International Renal Interest Society (IRIS) based on serum creatinine measurements. Studies of the pathogenesis of CKD by stage show disease characteristics, as follows:

Stage I: The remaining normal renal parenchyma (51%-75%) is significantly less than in geriatric controls. Interstitial inflammation consisted exclusively of lymphocytes in a regionally extensive distribution, similar for all stages, but the severity of inflammation in stage I (<25%) was greater than in young cats and less than later CKD stages. Cortical and medullary scarring (the presence of collagenous matrix visible with trichrome stain) was nearly absent (<25% in a single cat) at this stage and significantly less than all other stages. Tubular degeneration was mild, focal to scattered, which was significantly less severe than later stages (stages III and IV). Single-cell necrosis of tubular epithelial cells—characterized by loss of basement membrane adhesion, pyknotic to karyorrhectic nuclei, and shrunken, hypereosinophilic cytoplasm—was infrequent and significantly less than that observed in geriatric controls and other CKD stages. Global glomerulosclerosis—in which >75% of the capillary tuft was effaced by extracellular matrix—was observed less frequently in stage I than all other CKD stages but was greater than that observed in young cats. Other glomerular lesions and vascular lesions were absent. Blood chemistry in both dogs and cats shows normal blood creatinine or normal or mild increase blood SDMA.

Stage II: The amount of remaining normal parenchyma (51%-75%) and severity of inflammation (<25%) were similar to that in stage I cats. Interstitial inflammation consisted of primarily lymphocytes and plasma cells and less frequently macrophages and granulocytes. Cortical and medullary scarring was significantly greater than in controls or CKD stage I cats but not different from stage III. Interstitial lipid was present in most cats and was more frequent than in controls. Mild to moderate tubular degeneration was observed and was greater than in both controls and less than that in stages III and IV. Epithelial single-cell necrosis was greater than in young cats or stage I cats but similar to geriatric control cats. Global glomerulosclerosis was significantly greater than in controls and stage I cats but significantly less than in later stages. Other glomerular lesions were identified in a few cats including membranoproliferative glomerulonephritis (MPGN) and focal segmental glomerulosclerosis (FSGS), cystic glomerular atrophy, or mesangial expansion. Bowman's capsule thickening with or without parietal cell hypertrophy was present in most cats. Vascular lesions included fibrointimal hyperplasia, hyperplastic arteriolosclerosis, hyalinosis, and torturous vessels in regions of scarring; their prevalence was not statistically different among groups. In both dogs and cats, blood chemistry shows normal or mildly increased creatinine, mild renal azotemia (lower end of the range lies within reference ranges for creatinine for many laboratories, but the insensitivity of creatinine concentration as a screening test means that patients with creatinine values close to the upper reference limit often have excretory failure) and mildly increased SDMA. Clinical signs are usually mild or absent.

Stage III: Significantly less normal parenchyma remained at this stage (25%-50%) compared with stages I and II. Interstitial inflammation (25%-50%) was greater than earlier CKD stages and less than in stage IV cats and appeared as regionally extensive infiltrates of lymphocytes accompanied in half of cases by plasma cells, macrophages, and granulocytes. Severity of renal scarring (<25%) was similar between stage II and stage III cats; cortical scarring was significantly less compared with stage IV. Tubular degeneration was moderate to severe and significantly greater than in controls and stages I and II. Global glomerulosclerosis (note that sclerosis is generally considered to be secondary to chronic fibrosis) was greater than in controls and earlier stages but less than in stage IV cats. Thickening of Bowman's capsule and parietal cell hypertrophy often present. Hyperplastic arteriolosclerosis was observed more frequently than in young cats, but was not different from other stages.

Stage IV: Significantly less normal parenchyma compared to other stages. Inflammation affecting 51% to 75% of the tissue section was significantly greater than in all other groups and consisted of lymphocytes and plasma cells in most cats. Cortical scarring (25%-50%) was typically greater than medullary scarring (<25%); cortical scarring was significantly greater in stage IV than all other groups, while medullary scarring was significantly different only from controls and stage I cats. Regionally extensive scarring was most frequently encountered and was significantly different from controls and stages I and II. Interstitial lipid was present in all cats. Tubular degeneration, affecting entire nephrons, and single-cell necrosis of tubular epithelial cells were significantly more severe than in controls and earlier stages but similar to stage III cats. Tubular dilation and cysts were more prevalent than in stage I or Stage II, respectively. Global glomerulosclerosis was the most severe at this stage compared with all other groups. Most cats had at least one other glomerular lesion, including FSGS, glomerular hypertrophy, mesangial expansion, endothelial hypertrophy, MPGN pattern, and cystic glomerular hypertrophy; these were significantly more prevalent than in controls and stage II cats. Kidneys frequently contained fibrointimal hyperplasia but infrequently were affected by hyperplastic arteriolosclerosis. The prevalence of vascular lesions was not significantly different from controls or other stages. Blood chemistry shows moderate renal azotemia with elevated creatinine and SDMA. Many extrarenal signs may be present, but their extent and severity may vary. If signs are absent, the case could be considered as early Stage 3, while presence of many or marked systemic signs might justify classification as late Stage 3.

Normal parenchyma was unaffected by degeneration, atrophy, inflammation, or fibrosis was significantly less in later stages of CKD (ie, stages III and IV) compared with earlier stages (stages I and II) but similar between stages I and II. Interestingly, as little as 25% to 50% of the parenchyma was affected in the earlier stages of CKD, implying that even a mild degree of lesions could have functional significance. This is in contrast to the dogma that at least 75% of functional mass must be lost before clinical evidence of renal disease is evident. Blood chemistry shows increasingly elevated creatinine and SDMA, and there are increasing risks of systemic clinical signs and uremic crises.

Interstitial fibrosis and scarring, confirmed by Masson's trichrome stain, was statistically greater in stage IV compared with all other stages. Cats in stage IV were most likely to have 25% to 50% of their kidneys affected by scarring in comparison to ≤25% scarring in other stages. Interstitial fibrosis did not increase significantly between cats in stage II and III. This is in contrast to a previous study (which did not evaluate tissues stained with trichrome) in which interstitial fibrosis was the lesion that best correlated with severity of azotemia. This suggests that additional pathologic processes other than fibrosis are involved in disease progression and implies that initiation of any potential antifibrotic therapies in CKD cats should occur prior to stage IV, when irreversible fibrosis is most severe. This would be compatible with inflammation preceding and inducing fibrosis. However, patterns of scarring did not parallel that of interstitial inflammation, and a significant progression in scarring patterns from focal to regional to diffuse with increasing IRIS stage was not found. This suggests that instigators of fibrosis other than inflammation may be players in the progression of CKD and should be identified and evaluated as potential therapeutic targets.

A pathology summary from staging separate report shows that the severity of tubular degeneration, interstitial inflammation, fibrosis, and glomerulosclerosis was significantly greater in later stages of CKD compared with early stages of disease. Proteinuria was associated with increased severity of tubular degeneration, inflammation, fibrosis, tubular epithelial single-cell necrosis, and decreased normal parenchyma. Presence of hyperplastic arteriolosclerosis, fibrointimal hyperplasia, or other vascular lesions were not found to be significantly different between hypertensive and normotensive cats. The greater prevalence and severity of irreversible lesions in stage III and IV CKD implies that therapeutic interventions should be targeted at earlier stages of disease (McLeland, S. M. et al., Vet Pathol., 52(3):524-534 (2015)). Proteinuria, anemia, and hyperphosphatemia predict progression in feline CKD. These changes might reflect more progressive types of renal disease. Alternatively, they might reflect mechanisms of CKD progression such as tubular protein overload, hypoxia, and nephrocalcinosis (Chakrabarti, S. et al. J Vet Intern Med., 26:275-281 (2012)). Renal scarring encompasses interstitial fibrosis, which is an increase in extracellular matrix, as well as glomerulosclerosis and tubular atrophy. Tubulointerstitial changes, including fibrosis, are present in the early stages of feline CKD and become more severe in advanced disease. Collectively, these changes imply a loss of function and are considered, at least to date, irreversible. However, not all injury leads to irreversible damage. Replication and repair can lead to a return of normal function. Inflammation, edema, and tubular epithelial damage have the potential to resolve (ie, reversible) (McLeland, S. M. et al., Vet Pathol., 52(3):524-534 (2015)).

### 4. Etiology of CKD

The etiology of CKD is often unknown, but breed (purebreds at higher risk), age, and presence/severity of periodontal disease (1.5× more at risk than cats without periodontal disease) are major risk factors for the development of chronic kidney disease in domestic cats. The RVC retrospective study concluded that “These conditions are complex—meaning that there are many factors relating to genetics, lifestyle and environment that cumulatively determine whether an individual may develop either CKD or hypertension in their lifetime.”

In humans, the leading cause for end-stage renal failure is type 2 diabetes mellitus and hypertension. However, diabetic nephropathy has not been identified in cats, and renal lesions in diabetic cats have been no different from those in nondiabetic cats. In contrast to people and dogs, primary glomerulopathies with marked proteinuria are remarkably rare findings in cats. Although a variety of primary renal diseases have been implicated, the disease is idiopathic in most cats.

A variety of factors—including aging, ischemia, comorbid conditions, phosphorus overload, and routine vaccinations—have been implicated as factors that could contribute to the initiation of this disease in affected cats. Aging and stress seem to play an important role.

Other factors linked to renal disease include congenital malformation of the kidneys (birth defects), chronic bacterial infection of the kidneys with or without kidney stones (pyelonephritis), high blood pressure (hypertension), diseases associated with the immune system (glomerulonephritis, systemic lupus), and acute kidney disease (for example, poisoning with antifreeze that damages the kidneys can lead to chronic kidney disease).

Common clinical signs of CKD include drinking too much (polydipsia) and urinating large volumes of urine (polyuria), incontinence (leaking urine), especially at night, vomiting and/or diarrhea, lack of appetite and weight loss, general depression related to the elevation of waste products in the blood, anemia resulting in pale gums and weakness due to a low blood count, Gingivitis, and overall weakness from low blood potassium. Less common signs of CKD include weakened bones which can result in bone fractures, high blood pressure can lead to sudden blindness, itchy skin from calcium and phosphorous deposits and bleeding into the stomach or gut or bruising of skin. The most frequent morphologic diagnosis in cats with CKD is chronic tubulointerstitial nephritis and fibrosis, which are relatively nonspecific lesions.

### 5. Pathogenesis of CKD

Factors that are related to the progression of established CKD, which occurs in some but not all cats, include dietary phosphorus intake, magnitude of proteinuria, and anemia. Renal fibrosis, a common histologic feature of aged feline kidneys, interferes with the normal relationship between peritubular capillaries and renal tubules. Experimentally, renal ischemia results in morphologic changes similar to those observed in spontaneous CKD. Renal hypoxia, perhaps episodic, may play a role in the initiation and progression of this disease (Brown, C. A. et al., Vet Pathol., 53(2):309-326 (2016)).

Factors that are related to the progression of established CKD, which occurs in some but not all cats, include dietary phosphorus intake, magnitude of proteinuria, and anemia. Renal fibrosis, a common histologic feature of aged feline kidneys, interferes with the normal relationship between peritubular capillaries and renal tubules. Experimentally, renal ischemia results in morphologic changes similar to those observed in spontaneous CKD. Renal hypoxia, perhaps episodic, may play a role in the initiation and progression of this disease.

A recent publication comparing renal disease in wild and captive cheetahs showed that “renal medullary fibrosis was the only lesion associated with the likelihood of death being due to chronic renal disease, and cheetahs with this lesion were younger, on average, than cheetahs with other renal lesions” (Mitchell E. P, PLoS ONE, 13(3): e0194114 (2018)). These results suggest that age and renal medullary fibrosis are the primary factors influencing the pathogenesis of chronic renal disease in captive cheetahs. Apart from amyloidosis, these findings are analogous to those described in chronic renal disease in domestic cats, which is postulated to result primarily from repetitive hypoxic injury of renal tubules, mediated by age and stress.”

### B. Identification of Target Sequences

The present invention relates to a method for detecting the presence or amount of a target polynucleotide (nucleic acid sequence) from the host's response to CKD in a sample. The target polynucleotide is a virulence determinant. In a preferred embodiment, the target polynucleotide is miRNA. The invention is also directed to a method of detecting the presence of a disease or infection state in a mammal, by detecting the presence or amount of a target miRNA, wherein the presence or amount of the target miRNA identifies the disease state. Thus, the invention relates to diagnostic compositions and methods for detecting CKD. The sample containing the target miRNA may be tissue, collection of cells, cell lysate, body fluid, excretum, in vitro culture, purified polynucleotide, isolated polynucleotide, food sample, medical sample, agro-livestock sample, or environmental sample.

In another embodiment, the invention provides a method for capturing, detecting, and quantifying miRNA from its reverse transcribed cDNA. miRNA is extracted from the provided biological sample using commercially available miRNA specific extraction kits and the manufacturer's recommended protocol (e.g. Qiagen miRNeasy Serum/Plasma Kit). From the extracted miRNA, cDNA is reverse transcribed and amplified using commercially available miRNA to cDNA specific extraction kits and the manufacturer's recommended protocol (e.g. TaqMan Advanced miRNA cDNA Synthesis Kit). The resulting reverse transcribed cDNA of the miRNA may be captured and/or detected using the universal sequences added at both the 5′ and 3′ ends and the cDNA product may undergo universal pre-amplification and/or amplification using a single pair of universal forward and reverse primers. The relative expression levels of specific miRNA, which form part of the defined diagnostic panel, are inferred through the relative expression levels of their respective cDNA, i.e. detection by proxy. This can be performed via numerous traditional DNA detection methods, such as qPCR or Next Generation sequencing, or via newer multiplexing techniques such as beads capture technologies such as the Luminex xMAP system.

The invention described here utilizes large-scale identification of disrupted genes and the use of bioinformatics and AI to select mutants that could be characterized in animals.

C. Multiplex miRNA Profiling

The present invention uses multiplex miRNA profiling without RNA purification. Accuracy of miRNA profiling is enhanced when sample processing can be kept to a minimum, avoiding steps such as RNA purification that can introduce bias and inaccuracies. The present invention used a multiplex circulating miRNA assay that enables the profiling of a plurality of miRNAs in the same well directly from the sample, with no need for RNA purification. In one embodiment, the assay uses Luminex xMAP system beads, which enable the multiplex capture of miRNAs with picomolar sensitivity and high specificity. The Luminex xMAP beads functions with bespoke probes which contain three distinct functional regions: a complementary DNA section to the relevant DNA tag on the Luminex xMAP bead; an RNA region complimentary to the target miRNA; and a biotin fluorescent reporter tag. Detection is carried out using a Luminex LX200, or other compatible device, to detect miRNA molecules that emit fluorescence that is proportional to their abundance in the sample. Each miRNA that was used was given a unique bead region (up to 80 different regions were possible). The data that was obtained from the mixture of particles could then be attributed to the miRNAs by identification of the code.

The present invention uses multiplex miRNA relative expression profiling with machine learning and predictive classification analysis. Accuracy of miRNA expression profiling is enhanced when marker expression is analyzed relative to all other markers, as this allows detection of both increased and decreased expression, something not possible with simple threshold level analysis. The present invention used a RT-qPCR approach, with an option step of detection form synthesized cDNA to enhance sensitivity of detection. In one embodiment, the assay uses sample RNA extraction (e.g. Qigen miRNeasy kits), cDNA synthesis and amplification (e.g. TaqMan™ Advanced miRNA cDNA Synthesis Kit) followed by RT-qPCR detection with marker specific primers (e.g. TaqMan™ Advanced miRNA assays). Detection is carried out using a QuantStudio 5 Real-Time PCR Systems, or other RT-qPCR compatible device, to detect miRNA molecules that emit fluorescence that is proportional to their abundance in the samples. An alternate embodiment may utilize Luminex xMAP system beads, which enable the multiplex capture of miRNAs with picomolar sensitivity and high specificity. The Luminex xMAP beads function with bespoke probes which contain three distinct functional regions: a complementary DNA section to the relevant DNA tag on the Luminex xMAP bead; an RNA region complimentary to the target miRNA; and a biotin fluorescent reporter tag. Detection is carried out using a Luminex LX200, or other compatible device, to detect miRNA molecules that emit fluorescence that is proportional to their abundance in the sample. Each miRNA that was used was given a unique bead region (up to 80 different regions were possible). The data that was obtained from the mixture of particles could then be attributed to the miRNAs by identification of the code.

The disease is selected from the group consisting of CKD and related conditions.

The sample or blood sample refers to tissue or organ sample, blood, cell-free blood such as serum and plasma, urine, saliva, milk and cerebrospinal fluid sample.

From the results of the experiments below, a differentiation in expression levels of miRNA was identified when comparing healthy animals with animals that have CKD.

### D. Predictive Modelling

Provided herein are methods using predictive modelling to investigate the scope to use the miRNA profiles to predict the presence or absence of disease. A group of healthy and unhealthy animals were taken and tested to determine the level of miRNA expression in samples from these animals. The data obtained was then used to train the models.

Fifteen machine learning models were fitted and compared with the aim of obtaining the best predictions of the disease outcome. Formal assessment of performance was conducted by computing a number of performance statistics based on 5-time repeated 10-fold cross-validation. Cross-validation was useful to obtain more realistic model performance measures from the training data.

Data from the RT-qPCR analysis from each of the miRNA molecules from Table 1 was fitted to each of the models.

This disclosed invention utilized microRNA (miRNA) expression profile analysis to distinguish chronic kidney disease (CKD) cases from healthy controls in canine serum samples. A proprietary bioinformatic pipeline incorporating machine learning was used to optimize predictive classification models, achieving a 95% accuracy, 0.97 sensitivity, and 0.94 specificity, with a ROC AUC of 0.98. Additionally, principal component analysis (PCA) and clustering confirmed that CKD-associated miRNA profiles were distinct from controls but similar between urine and serum, promoting urine as an additional sample medium. The disclosed methods successfully demonstrated that miRNA biomarkers can differentiate CKD from healthy cases and are detectable in urine.

### E. Kits

Also provided herein is a kit for use in performing the method of the first aspect comprising means for determining the level of expression of each one of the following miRNA molecules: let-7b, miR-10a, miR-10b, miR-126, miR-130a-3p, miR-132, miR-135a, miR-135b, miR-143, miR-144, miR-146b, miR-150, miR-155, miR-16, miR-200a, miR-200b, miR-200c, miR-204, miR-206, miR-21, miR-214, miR-217, miR-21a, miR-223, miR-25, miR-26a, miR-27a, miR-29a, miR-29b, miR-29c, miR-30a, miR-30b, miR-30c, miR-30d, miR-30e, miR-34a, miR-382, miR-451, miR-486, and miR-802.

There is also provided a method of selecting a panel for use in disease diagnosis comprising the steps of: (a) selecting a group of miRNA molecules the differential expression of which may be associated with a disease condition; (b) applying one or more predictive classification algorithms to be able to predict the disease condition; and (c) using the one or more predictive classification algorithms to reduce the number of miRNAs in the panel to a minimum number to provide a panel of miRNAs that still produces a result.

### EXAMPLES

### Methods and Materials

This study included surplus blood samples from cases assessed during the study period. Blood samples were taken as clinically indicated testing. No blood was taken specifically for this study. Ethical approval was granted for the study at both sites, and consent obtained for research use of samples.

Sample collection and exclusion criteria: Blood samples (serum or plasma) were collected from unique cases. Additional sample data included various animal characteristics, including breed, age, sex, and neuter status for all samples. All CKD cases were assessed and confirmed by a specialist veterinarian or veterinary resident under the supervision of a Diplomate. Data from the physical examination and auscultation findings were retrieved by a retrospective review of medical records. Medication received at the time of presentation were recorded. Cases were categorized and staged. The 2nd generation NT-proBNP assay was used (IDEXX, Wetherby). Concentration of >900 pmol/L was defined as abnormal, based on the laboratory reference ranges. Results were dichotomized to normal or abnormal values (i.e. exceeding the respective reference range) in both control and CKD cases. Values above the reference ranges were considered to reflect cardiac disease, specifically CKD, since other renal and systemic conditions had been excluded. Any CKD cases that had history or clinical signs of concurrent disease were excluded from the study.

Clinical examination of CKD cases: Physical examination findings were recorded at the time of examination, and renal data retrieved.

Clinical examination of all CKD cases for the study were performed by one or more of the authors. The medication dogs were on at the time of the assessment noted, but medication subsequently prescribed as a result of the assessment and sampling was not included.

MicroRNA expression profiling: MicroRNAs included in the CKD-specific miRNA panel were selected through a review of manuscripts identified in a PubMed-based literature search which included CKD research in humans, dogs, cats and rodents. This was supplemented and adjusted using mirPath v3 database (Vlachos I S, et al., 1; 43(W1):W460-6 (2015)) to predict likely roles of microRNAs based on the pathology of mitral valve disease and activated disease pathways. This resulted in a sequence of 20 miRNAs noted to have altered expression during heart disease which were mapped to MIRBase for confirmation of sequences and nomenclature (Griffiths-Jones S. miRBase: The MicroRNA Sequence Database. In: MicroRNA Protocols [Internet]. New Jersey: Humana Press; 2006 [cited 2023 May 5]. p. 129-38) (Table 1). The most stable expressed miRNAs in an exploratory data set of canine and feline samples (n=556) were selected as normalizer miRNAs using Luminex. These included miRNAs previously suggested to be involved in canine cardiac disease but were found to have low variance in our dataset. Lastly, three off-species miRNAs were also included to act as background controls (Table 1). These sequences were used to design a custom 23-plex panel for the Luminex miRNA platform. For expression profiling, 50 μL aliquots of sera from each sample were incubated with Luminex beads and processed following the manufacturer's instructions with optimised hybridization, melt-off and capture temperatures. The mean florescence intensities (MFI) of miRNA-specific particles per sample was measured to quantify miRNA expression using a Novocyte flow cytometer and Novosampler Pro software (Agilent, Santa Clara, USA). Raw FCS files were exported to Fireplex Analysis Workbench 2.0.274 (Abcam, Cambridge, UK) and normalized expression values prepared using the ‘geomean’ function with the pre-selected normalisers identified in the preliminary dataset.

TABLE 1
Summary information for the profiling panel indicating mature sequence and
available predicted signaling pathways targeted by each miRNA (p < 0.05).

| SEQ | | | | SEQ | |
|---|---|---|---|---|---|
| ID NO: | miRNA | | miRNA Sequences | ID NO: | cDNA Sequences |
| 1 | let-7b | cfa-let- | TGAGGTAGTAGG | 50 | UGAGGUAGUAGG |
| | | 7b-5p | TTGTGTGGTT | | UUGUGUGGUU |
| 2 | miR- | cfa- | TACCCTGTAGATC | 51 | UACCCUGUAGAUC |
| | 10a | miR- | CGAATTTGT | | CGAAUUUGU |
| | | 10a-5p | | | |
| 3 | miR- | cfa- | CCCTGTAGAACC | 52 | CCCUGUAGAACCG |
| | 10b | miR- | GAATTTGTGT | | AAUUUGUGU |
| | | 10b-5p | | | |
| 4 | miR- | cfa- | CATTATTACTTTT | 53 | CAUUAUUACUUU |
| | 126 | miR- | GGTACGCG | | UGGUACGCG |
| | | 126-5p | | | |
| 5 | miR- | cfa- | CAGTGCAATGTTA | 54 | CAGUGCAAUGUU |
| | 130a | miR- | AAAGGGCAT | | AAAAGGGCAU |
| | | 130a-3p | | | |
| 6 | miR- | cfa- | TAACAGTCTACA | 55 | UAACAGUCUACAG |
| | 132 | miR- | GCCATGGTCGC | | CCAUGGUCGC |
| | | 132-3p | | | |
| 7 | miR- | cfa- | TATGGCTTTTTAT | 56 | UAUGGCUUUUUA |
| | 135a | miR- | TCCTATGTGA | | UUCCUAUGUGA |
| | | 135a-5p | | | |
| 8 | miR- | cfa- | TATGGCTTTTCAT | 57 | UAUGGCUUUUCA |
| | 135b | miR- | TCCTATGTGA | | UUCCUAUGUGA |
| | | 135b-5p | | | |
| 9 | miR- | cfa- | TGAGATGAAGCA | 58 | UGAGAUGAAGCA |
| | 143 | miR- | CTGTAGCTC | | CUGUAGCUC |
| | | 143-3p | | | |
| 10 | miR- | cfa- | TACAGTATAGAT | 59 | UACAGUAUAGAU |
| | 144 | miR- | GATGTACTAG | | GAUGUACUAG |
| | | 144-3p | | | |
| 11 | miR- | cfa- | TGAGAACTGAAT | 60 | UGAGAACUGAAU |
| | 146b | miR- | TCCATAGGCT | | UCCAUAGGCU |
| | | 146b-5p | | | |
| 12 | miR- | cfa- | TCTCCCAACCCTT | 61 | UCUCCCAACCCUU |
| | 150 | miR- | GTACCAGTG | | GUACCAGUG |
| | | 150-5p | | | |
| 13 | miR- | cfa- | TTAATGCTAATCG | 62 | UUAAUGCUAAUC |
| | 155 | miR- | TGATAGGGGT | | GUGAUAGGGGU |
| | | 155-5p | | | |
| 14 | miR-16 | cfa- | TAGCAGCACGTA | 63 | UAGCAGCACGUAA |
| | | miR- | AATATTGGCG | | AUAUUGGCG |
| | | 16-5p | | | |
| 15 | miR- | cfa- | CATCTTACCGGAC | 64 | CAUCUUACCGGAC |
| | 200a | miR- | AGTGCTGGA | | AGUGCUGGA |
| | | 200a-5p | | | |
| 16 | miR- | cfa- | CATCTTACTGGGC | 65 | CAUCUUACUGGGC |
| | 200b | miR- | AGCATTGGA | | AGCAUUGGA |
| | | 200b-5p | | | |
| 17 | miR- | cfa- | TAATACTGCCGG | 66 | UAAUACUGCCGGG |
| | 200c | miR- | GTAATGATGGA | | UAAUGAUGGA |
| | | 200c-3p | | | |
| 18 | miR- | cfa- | TTCCCTTTGTCAT | 67 | UUCCCUUUGUCAU |
| | 204 | miR- | CCTATGCCT | | CCUAUGCCU |
| | | 204-5p | | | |
| 19 | miR- | cfa- | TGGAATGTAAGG | 68 | UGGAAUGUAAGG |
| | 206 | miR- | AAGTGTGTGG | | AAGUGUGUGG |
| | | 206-3p | | | |
| 20 | miR-21 | cfa- | TAGCTTATCAGAC | 69 | UAGCUUAUCAGAC |
| | | miR- | TGATGTTGA | | UGAUGUUGA |
| | | 21-3p | | | |
| 21 | miR- | cfa- | ACAGCAGGCACA | 70 | ACAGCAGGCACAG |
| | 214 | miR- | GACAGGCAGT | | ACAGGCAGU |
| | | 214-3p | | | |
| 22 | miR- | cfa- | TACTGCATCAGG | 71 | UACUGCAUCAGGA |
| | 217 | miR- | AACTGATTGGAT | | ACUGAUUGGAU |
| | | 217-5p | | | |
| 23 | miR- | cfa- | TAGCTTATCAGAC | 72 | UAGCUUAUCAGAC |
| | 21a | miR- | TGATGTTGA | | UGAUGUUGA |
| | | 21-5p | | | |
| 24 | miR- | cfa- | TGTCAGTTTGTCA | 73 | UGUCAGUUUGUC |
| | 223 | miR- | AATACCCC | | AAAUACCCC |
| | | 223-3p | | | |
| 25 | miR-25 | cfa- | CATTGCACTTGTC | 74 | CAUUGCACUUGUC |
| | | miR- | TCGGTCTGA | | UCGGUCUGA |
| | | 25-3p | | | |
| 26 | miR- | cfa- | TTCAAGTAATCCA | 75 | UUCAAGUAAUCCA |
| | 26a | miR- | GGATAGGCT | | GGAUAGGCU |
| | | 26a-5p | | | |
| 27 | miR- | cfa- | TTCACAGTGGCTA | 76 | UUCACAGUGGCUA |
| | 27a | miR- | AGTTCCG | | AGUUCCG |
| | | 27a-3p | | | |
| 28 | miR- | cfa- | TAGCACCATCTGA | 77 | UAGCACCAUCUGA |
| | 29a | miR- | AATCGGTTA | | AAUCGGUUA |
| | | 29a-3p | | | |
| 29 | miR- | cfa- | TAGCACCATTTGA | 78 | UAGCACCAUUUGA |
| | 29b | miR- | AATCAGTGTT | | AAUCAGUGUU |
| | | 29b-3p | | | |
| 30 | miR- | cfa- | TAGCACCATTTGA | 79 | UAGCACCAUUUGA |
| | 29c | miR- | AATCGGTTA | | AAUCGGUUA |
| | | 29c-3p | | | |
| 31 | miR- | cfa- | TGTAAACATCCTC | 80 | UGUAAACAUCCUC |
| | 30a | miR- | GACTGGAAGC | | GACUGGAAGC |
| | | 30a-5p | | | |
| 32 | miR- | cfa- | TGTAAACATCCTA | 81 | UGUAAACAUCCUA |
| | 30b | miR- | CACTCAGCT | | CACUCAGCU |
| | | 30b-5p | | | |
| 33 | miR- | cfa- | TGTAAACATCCTA | 82 | UGUAAACAUCCUA |
| | 30c | miR- | CACTCAGCT | | CACUCAGCU |
| | | 30c-5p | | | |
| 34 | miR- | cfa- | TGTAAACATCCCC | 83 | UGUAAACAUCCCC |
| | 30d | miR- | GACTGGAAGCT | | GACUGGAAGCU |
| | | 30d-5p | | | |
| 35 | miR- | cfa- | CTTTCAGTCGGAT | 84 | CUUUCAGUCGGAU |
| | 30e | miR- | GTTTACAGC | | GUUUACAGC |
| | | 30e-3p | | | |
| 36 | miR- | cfa- | TGGCAGTGTCTTA | 85 | UGGCAGUGUCUU |
| | 34a | miR- | GCTGGTTGT | | AGCUGGUUGU |
| | | 34a-5p | | | |
| 37 | miR- | cfa- | AATCATTCACGG | 86 | AAUCAUUCACGGA |
| | 382 | miR- | ACAACACTTT | | CAACACUUU |
| | | 382-3p | | | |
| 38 | miR- | cfa- | AAACCGTTACCAT | 87 | AAACCGUUACCAU |
| | 451 | miR- | TACTGAGTT | | UACUGAGUU |
| | | 451-5p | | | |
| 39 | miR- | cfa- | TCCTGTACTGAGC | 88 | UCCUGUACUGAGC |
| | 486 | miR- | TGCCCCGA | | UGCCCCGA |
| | | 486-5p | | | |
| 40 | miR- | cfa- | CAGTAACAAAGA | 89 | CAGUAACAAAGA |
| | 802 | miR- | TTCATCCTTGT | | UUCAUCCUUGU |
| | | 802-16 | | | |

Normalizers

| | | | | | |
|---|---|---|---|---|---|
| 41 | miR-16 | cfa- | TAGCAGCACGTA | 90 | UAGCAGCACGUAA |
| | | miR- | AATATTGGCG | | AUAUUGGCG |
| | | 16-5p | | | |
| 42 | U6 | U6 | CTCGCTTCGGCAG | 91 | CUCGCUUCGGCAG |
| | snRNA | snRNA | CACATATAC | | CACAUAUAC |
| 43 | cel- | cel- | TCACCGGGTGTA | 92 | UCACCGGGUGUAA |
| | miR-39 | miR- | AATCAGCTTG | | AUCAGCUUG |
| | | 39-3p | | | |
| 44 | RNU6B | RNU6B | CGCAAGGATGAC | 93 | CGCAAGGAUGACA |
| | | | ACGCAAATTCGT | | CGCAAAUUCGU |
| 45 | miR- | cfa- | TCGAGGAGCTCA | 94 | UCGAGGAGCUCAC |
| | 151 | miR- | CAGTCTAGT | | AGUCUAGU |
| | | 151-5p | | | |
| 47 | miR- | cfa- | CAAAGAATTCTCC | 95 | CAAAGAAUUCUCC |
| | 186 | miR- | TTTTGGGCT | | UUUUGGGCU |
| | | 186-5p | | | |
| 48 | miR- | cfa- | TTCAAGTAATTCA | 96 | UUCAAGUAAUUC |
| | 26b | miR- | GGATAGGTT | | AGGAUAGGUU |
| | | 26b-5p | | | |
| 49 | miR-28 | cfa- | CACTAGATTGTGA | 97 | CACUAGAUUGUG |
| | | miR- | GCTCCTGGA | | AGCUCCUGGA |
| | | 28-3p | | | |

Data preparation and exploration: The data consisting of miRNA expression profiles issued from healthy controls and CKD cases were processed by Luminex. Each miRNA profile was formed by measuring the normalized mean fluorescence intensity (MFI) of the miRNAs common across the two data batches. For each miRNA profile, MFIs were standardised by a centred log-ratio transformation applied to each sample to handle the compositionality of the quantification of miRNA molecules derived from varying sequencing library sizes across samples (Fernandes, A. D. et al., Microbiome, 2, 15 (2014)). To manage variation due to batch effects, weighted PLS-DA-batch correction method is applied, as proposed in Wang 2023 (Yiwen Wang, Kim-Anh Lê Cao, Briefings in Bioinformatics, Volume 24, Issue 2, (2023). This method was specifically designed for an unbalanced batch x disease status setting.

Predictive classification modelling: After preliminary investigation and comparative assessment of alternative statistical and machine learning approaches to select an optimal predictive modelling formulation, penalised logistic regression (PLR) models (Park M Y, Hastie T., Biostatistics, 9(1):30-50 (2008); Hastie, T., et al., (2001)), The Elements of Statistical Learning, Springer New York Inc., New York, NY, USA) are fitted to mean-centred data for the purpose of predictive classification of samples into CKD cases or healthy status; and (2) early stage CKD or late stage CKD. Formally, given a 2-class response variable y, taking values 1 (positive status) with probability p and 0 (negative status) with probability 1−p, and the vector of processed miRNA signals (and possibly other covariates) acting as predictors x=(x₁, . . . , xₚ), a logistic regression model of the form:

$$ln \frac{p}{1 - p} = {β}_{0} + {\sum }_{j = 1}^{p} {β}_{j} {x}_{j} .$$

was established. Using the maximum likelihood estimation method, PLR model coefficient estimates (\hat{β}₀, \hat{β}₁, . . . \hat{β}ₚ) were obtained by maximizing the penalized log-likelihood function:

$$max \{ {\sum }_{i = 1}^{n} [ {y}_{i} ( {β}_{0} + {\sum }_{j = 1}^{p} {β}_{j} {x}_{i j} ) - ln ( 1 + {e}^{{β}_{0} + {\sum }_{j = 1}^{p} {β}_{j} {x}_{i j}} ) ] - λ {\sum }_{j = 1}^{p} {\vert {β}_{j} \vert}^{2} \} ,$$

where n refers to the number of samples and λ is the penalty parameter, so that the coefficients less contributing to the prediction of the outcome were shrunk toward zero. Including such a penalty aid in preventing overfitting, favoring model unbiasedness, sparsity, and a stable fit with large numbers of predictors, typically affected by multicollinearity. Given the model estimates, the predicted status probabilities for a sample were obtained using:

$$\hat{p} = \frac{1}{1 + {e}^{- ( {\hat{β}}_{0} + {\hat{β}}_{1} {x}_{1} + … + {\hat{β}}_{p} {x}_{p} )}} ,$$

with a sample being allocated the status with the highest probability. Tuning to determine the level of shrinkage λ, parameter fitting by maximum likelihood, and performance assessment were all embedded into a 10-fold cross-validation (CV) pipeline. That is, the input data were randomly partitioned into ten folds, with nine folds used to train the model and one-fold used as validation set sequentially. This randomization was repeated 5 times. Performance metrics included overall accuracy, area under the receiver operating curve (AUC-ROC), sensitivity, specificity and the F1 score (regarded as fairer assessment of accuracy in case of unbalanced classes) (Hastie, T. et al., (2001), The Elements of Statistical Learning, Springer New York Inc., New York, NY, USA). All these metrics ranged in [0,1], with values closer to 1 indicating better performance. They were measured against each validation set and averaged across CV runs to assess how the model might perform when asked to predict from independent blind samples. These measures were accompanied by 95% confidence intervals (CI) where available. The model training pipeline and all data analyses and graphical representations were set up and conducted on the R system for statistical computing v4.2.1 (R Core Team. R: A language and environment for statistical computing. R Foundation for Statistical Computing, Vienna, Austria).

Samples: CKD cases. A total of 50 canine serum samples and 50 canine urine samples from CKD cases which were at IRIS stage 2 or 3 were made available by the Company. These samples were taken from a preexisting study and were collected based on the following criteria.

### Inclusion Criteria

1. Dogs with CKD of IRIS stages II or III based on presence of inadequately concentrated urine (urine specific gravity [USG]<1.030) and at least one of the following:
   a. renal proteinuria (urine protein/creatinine ratio [UPC]>0.5),
   b. ultrasonographic renal abnormalities (decreased corticomedullary distinction, small irregular kidneys, or both),
   c. serum creatinine concentration ≥1.4 mg/dL in the absence of other diseases likely to cause polyuria or polydipsia.

### Exclusion Criteria

1. Dogs less than one year of age or weighing <5 kg at enrolment.
2. Dogs that required medical hospitalization in the two weeks immediately prior to enrolment.
3. Dogs diagnosed with:
   a. primary hyperparathyroidism or primary hypoparathyroidism,
   b. protein-losing enteropathy,
   c. polycystic kidney disease,
   d. hypercalcemia of non-renal or non-renal secondary hyperparathyroid origin,
   e. pyelonephritis,
   f. pre- or post-renal azotemia such as obstructive lower urinary tract disease/urolithiasis,
   g. urinary tract infection,
   h. any malignant or suspected malignant neoplasia.

### Control Cases

A total of 45 canine serum samples control animals with blood and urine parameters consistent with normal kidney function as per the IRIS guidelines, and did not fall within the exclusion criteria outlined above.

### Methods

Based on previous research and existing background IP by MI:RNA, the samples were screened by qPCR for a panel of microRNA markers specific to renal pathology that were selected from a broad literature search. The expression profile of these markers was quantified by qPCR, and the results analyzed using MI:RNA's proprietary bioinformatic pipeline that uses machine learning to train various predictive classification algorithms. The optimal algorithm for the training data set was identified using various performance metrics (accuracy, ROC AUC, sensitivity and specificity). Tenfold cross-validation was used to reduce overfitting during training.

The final qPCR miRNA data (45 canine control serum, 39 CKD case canine serum, 40 CKD case canine urine) were normalized using two endogenous miRNA markers that showed low variability in the training data set (CoV ≈0.5). Optimization of miRNA profile data to account for missing data provided a maximal informative data set of five potential biomarker miRNAs across 27 controls and 32 CKD samples (urine and serum). miRNA profiles for CKD patients prepared from urine or serum samples were highly similar. As such, CKD samples were grouped for subsequent analysis. Fifteen machine learning algorithms were trained with 10-fold cross validation across five repeats. Model classification accuracy was assessed using ROC AUC, overall accuracy, sensitivity, and specificity. Variable importance was estimated using individual ROC curve analysis.

### Example 1: Differentiating miRNA Serum Profiles Between Healthy and CKD Cases

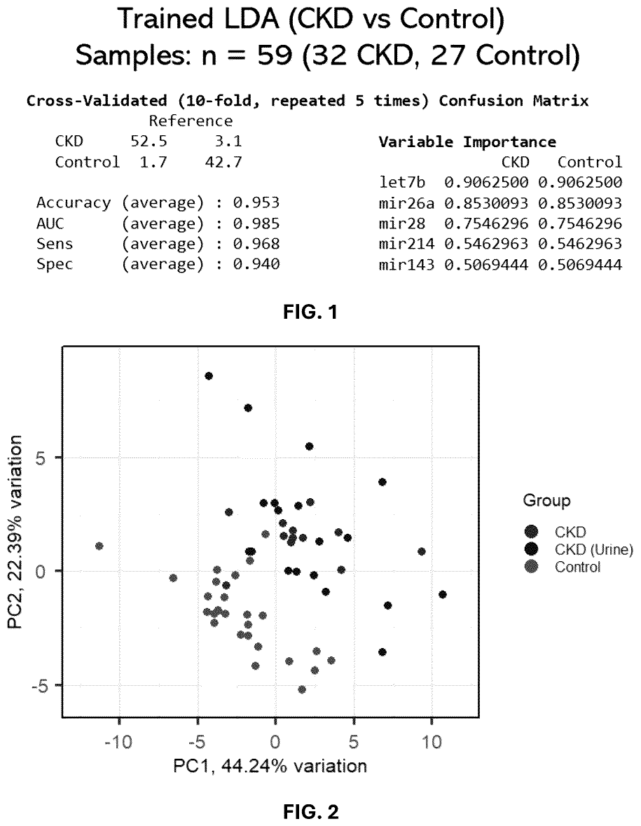

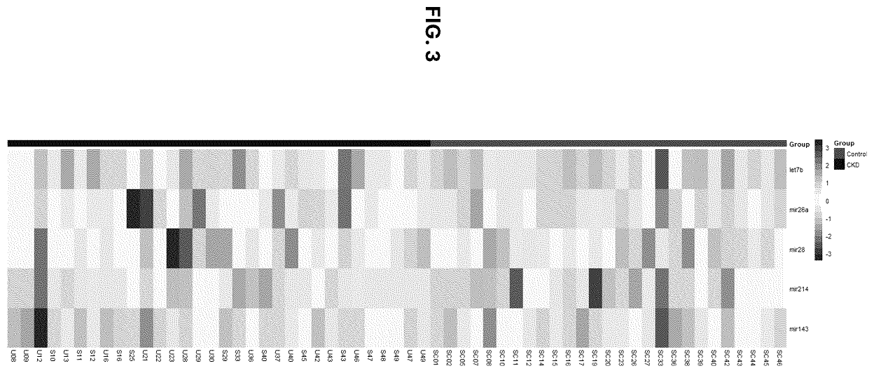

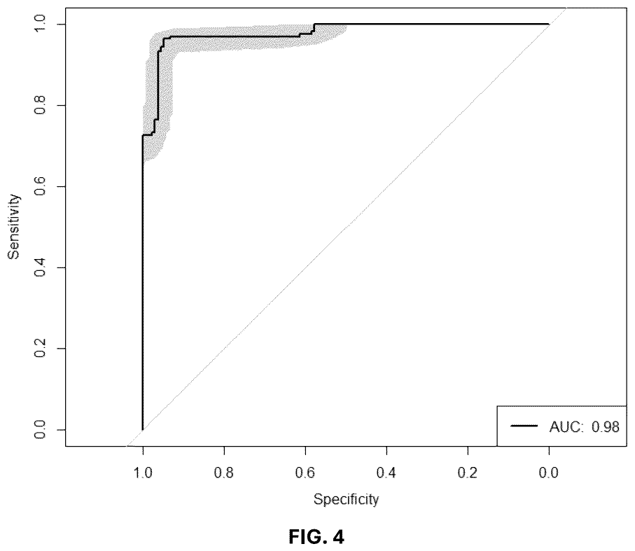

FIG. 1 shows the latent Dirichlet allocation (LDA) model statistics for Canine CKD versus control samples. Canine serum miRNA profiles of control and CKD samples separate on the second axis of a principal component analysis (PCA) (FIG. 2). Differences can also be observed on a heatmap of miRNA expression showing expression profiles for the most influential markers, let7b, mir26a, mir28, mir214, and mir143 (FIG. 3). CKD samples prepared from urine or blood could not be distinguished by PCA or k-means clustering. The most accurate model for binary classification (control vs CKD) provided an accuracy 95%, sensitivity 0.97 and specificity 0.94. Estimates of the 95% confidence intervals for the ROC AUC using the LDA model indicated that it was discriminative for the training data (ROC AUC=0.98) (FIG. 4). The panel and model show the ability to differentiate between health and CKD cases. miRNA CKD targets are detectable in urine, and cluster with CKD serum profiles, distinct from control miRNA profiles.

### Example 2: RT-qPCR-Based miRNA Profiling with Machine Learning Optimisation: Feline CKD

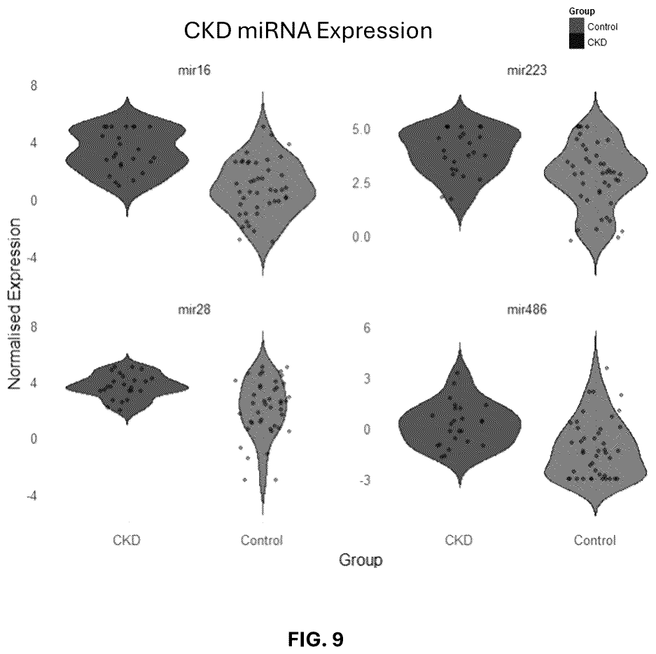

In total, 100 samples (50 controls and 50 IRIS stage 2/3 feline CKD cases) were reverse transcribed and assayed using TaqMan Advanced miRNA cDNA Synthesis kits specific to each miRNA. miRNA with 20%>missing data or where the duplicates Ct values were more than 2 SD were removed. miRNA Data were z-score normalised per sample to identify a marker with low variability and high amplification success suitable for normalisation (mir144). Data for the remaining miRNAs with 80% amplification rates (mir16, mir223, mir28, mir486) were then normalised using delta-Ct against the normaliser Ct and Yeo-Johnson transformed (FIG. 9). Twelve machine learning algorithms suitable for low feature number were trained with 10-fold cross validation across five repeats. Model classification accuracy was assessed using ROC AUC, overall accuracy, sensitivity, and specificity.

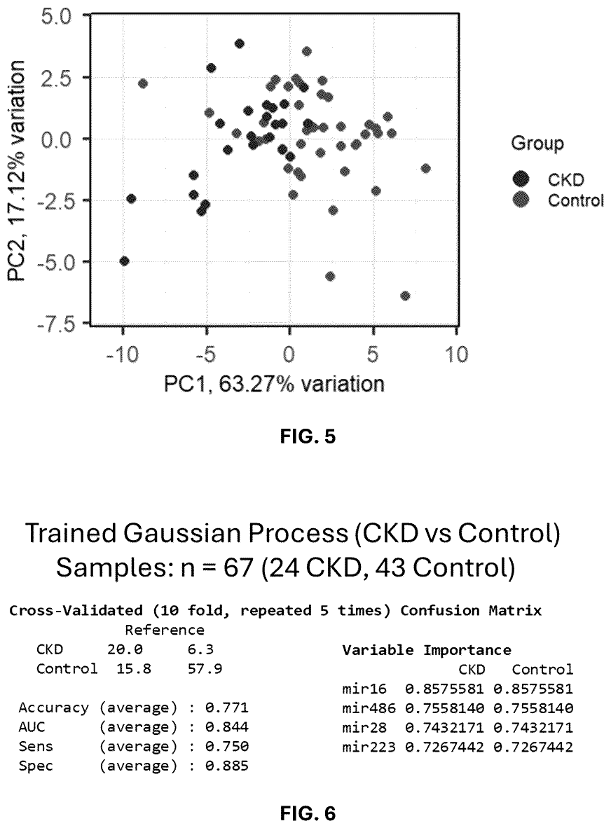

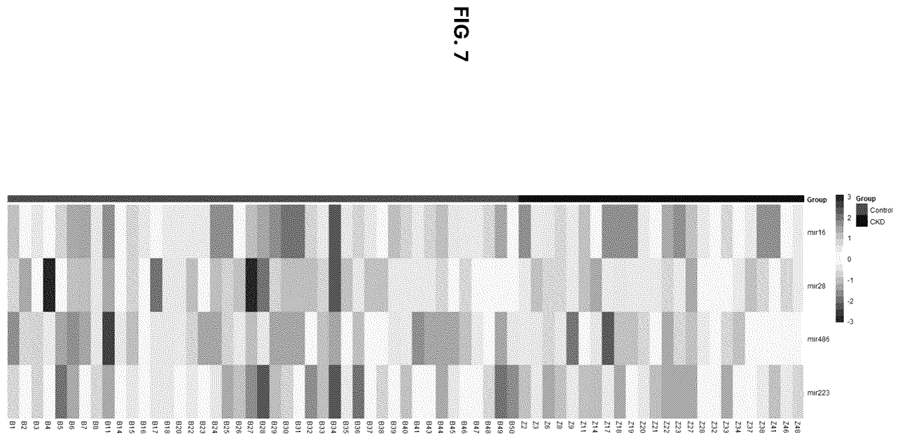

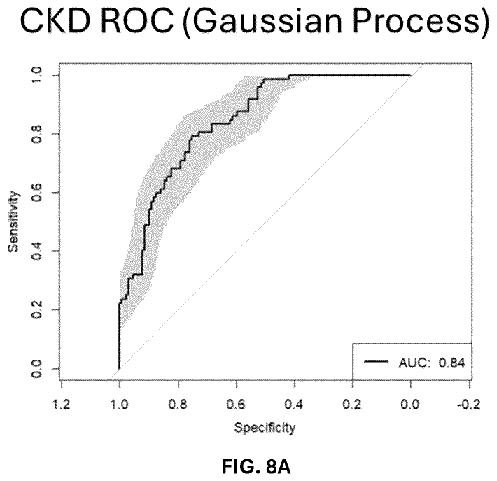

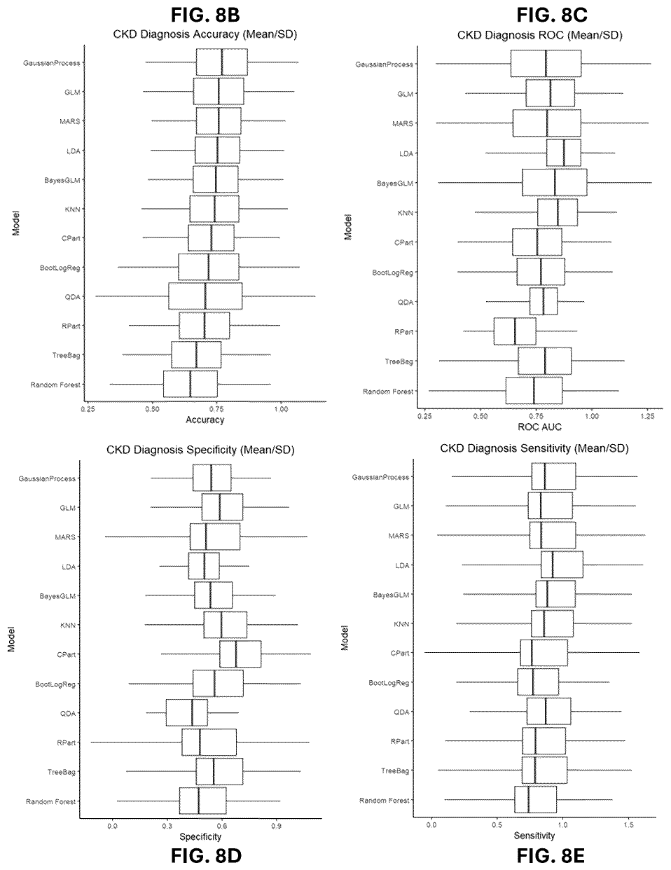

After filtering for samples with missing data across the miRNAs with >80% amplification rates, 24 CKD and 43 control samples remained. After normalisation, the expression profiles could be separated by disease status using PCA (FIG. 5) and there was some visible distinction between controls and CKD samples in a heatmap for miRNA expression (FIG. 7). The most accurate model for binary classification (control vs CKD) was a Gaussian process using a radial basis function kernel), with an overall accuracy 78%, 0.84 ROC AUC, sensitivity 0.75 and specificity 0.89 (FIG. 6 and FIG. 8A-8D). Estimates of the 95% confidence intervals for the ROC AUC using the Gaussian process model suggest that it is discriminatory in the training data (FIG. 8A).

Initial testing with a Gaussian process model trained using canine CKD data and the same miRNAs suggest that a different species model was partially discriminatory (accuracy=69%).

RT-qPCR miRNA profiling using the TaqMan Advanced miRNA cDNA Synthesis system can provide discriminatory information for diagnosing feline CKD in the training data set using models trained with machine learning. Each species appears to require a distinct model for optimal accuracy, likely reflecting differences in pathology between feline and canine CKD.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art were included within the invention defined by the claims.

What is claimed is:

1. A method for differentially assessing and diagnosing a diseased state of chronic kidney disease (CKD) in a subject, comprising the steps of:
(a) obtaining a sample from the subject;
(b) isolating miRNA molecules within a sample from a subject;
(c) amplifying the cDNA molecules to a detectable concentration;
(d) probing for the cDNA molecules complimentary to the desired miRNA markers;
(e) determining a level of expression of the miRNA molecules within a sample from a subject by the level of cDNA molecules probed for the desired miRNA markers; and
(f) using one or more Artificial Intelligence (AI) model to predict the disease condition of the subject;
wherein the one or more AI model compares the level of expression of each cDNA molecule with at least one pre-determined reference level cDNA molecule characteristic of a non-diseased subject wherein a deviation of the level of expression of said cDNA molecule in comparison with the at least one reference level cDNA molecule allows for the diagnosis and/or prognosis of CKD.

2. The method according to claim 1, wherein the cDNA molecule may also be a reverse compliment cDNA.

3. The method according to claim 1, wherein the miRNA molecules comprise a panel of reference miRNAs having at least one miRNA selected from a group consisting of nucleic acid sequence having at least 95%, 97%, 98% or 99% sequence identity to SEQ ID NO: 1, 2, 3, 4, 5, 6, 7, 8, 9, 10, 11, 12, 13, 14, 15, 16, 17, 18, 19, 20, 21, 22, 23, 24, 25, 26, 26, 28, 29, 30, 31, 32, 33, 34, 35, 36, 37, 38, 39, 40, or combinations thereof, the miRNA molecules having a reverse compliment cDNA with at least 99% sequence identity to SEQ ID NO: 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or combinations thereof.

4. The method according to claim 3, wherein the at least one miRNA molecule is mir144 having at least 99% sequence identity to SEQ ID NO:10, the miRNA molecules having a reverse compliment cDNA with at least 99% sequence identity to SEQ ID NO: 59.

5. The method according to claim 1, wherein the miRNA molecules comprise a panel of reference miRNAs having at least five miRNAs selected from a group consisting of nucleic acid sequence having at least 95%, 97%, 98% or 99% sequence identity to SEQ ID NO: 1, 2, 3, 4, 5, 6, 7, 8, 9, 10, 11, 12, 13, 14, 15, 16, 17, 18, 19, 20, 21, 22, 23, 24, 25, 26, 26, 28, 29, 30, 31, 32, 33, 34, 35, 36, 37, 38, 39, 40, or combinations thereof, the miRNA molecules having a reverse compliment cDNA with at least 99% sequence identity to SEQ ID NO: 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or combinations thereof.

6. The method according to claim 5, wherein the at least five miRNAs are mir144, mir16, mir223, mir28, mir486 having at least 99% sequence identity to SEQ ID NO:10, 14, 24, 49, and 39, respectively, the miRNA molecules having a reverse compliment cDNA with at least 99% sequence identity to SEQ ID NO: 59, 63, 73, 89, and 88, respectively.

7. The method according to claim 1, wherein the miRNA molecules comprise a panel of reference miRNAs having at least nine miRNAs selected from a group consisting of nucleic acid sequence having at least 95%, 97%, 98% or 99% sequence identity to SEQ ID NO: 1, 2, 3, 4, 5, 6, 7, 8, 9, 10, 11, 12, 13, 14, 15, 16, 17, 18, 19, 20, 21, 22, 23, 24, 25, 26, 26, 28, 29, 30, 31, 32, 33, 34, 35, 36, 37, 38, 39, 40, or combinations thereof, the miRNA molecules having a reverse compliment cDNA with at least 99% sequence identity to SEQ ID NO: 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or combinations thereof.

8. The method according to claim 7, wherein the at least nine miRNAs are let7b, mir26a, mir214, mir143, mir144, mir16, mir223, mir28, mir486 having at least 99% sequence identity to SEQ ID NO: 1, 26, 21, 9, 10, 14, 24, 49, and 39, respectively, the miRNA molecules having a reverse compliment cDNA with at least 99% sequence identity to SEQ ID NO: 50, 75, 70, 58, 59, 63, 73, 89, and 88, respectively.

9. The method according to claim 1, wherein the method further comprises the use of at least one normalizer and/or control miRNA molecule selected from a group consisting of nucleic acid sequence having at least 95%, 97%, 98% or 99% sequence identity to SEQ ID NO: 41, 42, 43, 44, 45, 46, 47, 48, and 49, the miRNA molecules having a reverse compliment cDNA with at least 99% sequence identity to SEQ ID NO: 90, 91, 92, 93, 94, 95, 96, and 97, wherein the normalizer or control miRNA molecule is an off-species control miRNA molecule.

10. The method according to claim 1, wherein the method further comprises the step of using a machine learning algorithm for predictive modelling, and
wherein the method comprises the use of a combination of AI models.

11. The method according to claim 1, wherein the subject is a mammal.

12. The method according to claim 1, wherein the subject is a dog or cat.

13. The method according to claim 1, wherein the sample is a biofluid selected from the group consisting of blood, urine, milk, tissue fluid, saliva, cerebrospinal fluid (CSF), feces or another biofluid.

14. A kit for use in performing the method of claim 1 comprising means for determining the level of expression of miRNA molecules selected from a miRNA panel having at least nine miRNA molecules having at least 95%, 97%, 98% or 99% sequence identity to SEQ ID NO: 1, 2, 3, 4, 5, 6, 7, 8, 9, 10, 11, 12, 13, 14, 15, 16, 17, 18, 19, 20, 21, 22, 23, 24, 25, 26, 26, 28, 29, 30, 31, 32, 33, 34, 35, 36, 37, 38, 39, 40, or combinations thereof, the miRNA molecules having a reverse compliment cDNA with at least 99% sequence identity to SEQ ID NO: 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or combinations thereof.

15. The kit according to claim 14, wherein the at least nine miRNA molecules are let7b, mir26a, mir214, mir143, mir144, mir16, mir223, mir28, mir486 having at least 99% sequence identity to SEQ ID NO:1, 26, 21, 9, 10, 14, 24, 49, and 39, respectively, the miRNA molecules having a reverse compliment cDNA with at least 99% sequence identity to SEQ ID NO: 50, 75, 70, 58, 59, 63, 73, 89, and 88, respectively.

16. The kit according to claim 14 comprising a miRNA panel having at least five miRNA molecules having at least 95%, 97%, 98% or 99% sequence identity to SEQ ID NO: 1, 2, 3, 4, 5, 6, 7, 8, 9, 10, 11, 12, 13, 14, 15, 16, 17, 18, 19, 20, 21, 22, 23, 24, 25, 26, 26, 28, 29, 30, 31, 32, 33, 34, 35, 36, 37, 38, 39, 40, or combinations thereof, the miRNA molecules having a reverse compliment cDNA with at least 99% sequence identity to SEQ ID NO: 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or combinations thereof.

17. The kit according to claim 16, wherein the at least five miRNAs are mir144, mir16, mir223, mir28, mir486 having at least 99% sequence identity to SEQ ID NO:10, 14, 24, 49, and 39, respectively, the miRNA molecules having a reverse compliment cDNA with at least 99% sequence identity to SEQ ID NO: 59, 63, 73, 89, and 88, respectively.

18. The kit according to claim 14 comprising a miRNA panel having at least 1 miRNA molecule having at least 95%, 97%, 98% or 99% sequence identity to SEQ ID NO: 1, 2, 3, 4, 5, 6, 7, 8, 9, 10, 11, 12, 13, 14, 15, 16, 17, 18, 19, 20, 21, 22, 23, 24, 25, 26, 26, 28, 29, 30, 31, 32, 33, 34, 35, 36, 37, 38, 39, 40, or combinations thereof, the miRNA molecules having a reverse compliment cDNA with at least 99% sequence identity to SEQ ID NO: 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or combinations thereof.

19. The kit according to claim 18, wherein the at least one miRNA is mir144 having at least 99% sequence identity to SEQ ID NO:10, the miRNA molecules having a reverse compliment cDNA with at least 99% sequence identity to SEQ ID NO: 59.

20. A method of selecting a panel for use in disease diagnosis comprising the steps of:
(a) selecting a group of miRNA molecules the differential expression of which may be associated with a disease condition;
(b) predicting the disease condition based on a deviation of the level of expression of said miRNA molecules from step (a) and (b); and
(c) reducing the number of miRNAs in the panel to a minimum number to provide a panel of miRNAs that still produces a result;
wherein the disease is CKD.


SEQUENCE LISTING (97 sequences)

SEQ ID NO: 1 - DNA, 22 bp, synthetic construct
  source 1..22: mol_type = other DNA
tgaggtagtaggttgtgtggtt

SEQ ID NO: 2 - DNA, 22 bp, synthetic construct
  source 1..22: mol_type = other DNA
taccctgtagatccgaatttgt

SEQ ID NO: 3 - DNA, 22 bp, synthetic construct
  source 1..22: mol_type = other DNA
ccctgtagaaccgaatttgtgt

SEQ ID NO: 4 - DNA, 21 bp, synthetic construct
  source 1..21: mol_type = other DNA
cattattacttttggtacgcg

SEQ ID NO: 5 - DNA, 22 bp, synthetic construct
  source 1..22: mol_type = other DNA
cagtgcaatgttaaaagggcat

SEQ ID NO: 6 - DNA, 23 bp, synthetic construct
  source 1..23: mol_type = other DNA
taacagtctacagccatggtcgc

SEQ ID NO: 7 - DNA, 23 bp, synthetic construct
  source 1..23: mol_type = other DNA
tatggctttttattcctatgtga

SEQ ID NO: 8 - DNA, 23 bp, synthetic construct
  source 1..23: mol_type = other DNA
tatggcttttcattcctatgtga

SEQ ID NO: 9 - DNA, 21 bp, synthetic construct
  source 1..21: mol_type = other DNA
tgagatgaagcactgtagctc

SEQ ID NO: 10 - DNA, 22 bp, synthetic construct
  source 1..22: mol_type = other DNA
tacagtatagatgatgtactag

SEQ ID NO: 11 - DNA, 22 bp, synthetic construct
  source 1..22: mol_type = other DNA
tgagaactgaattccataggct

SEQ ID NO: 12 - DNA, 22 bp, synthetic construct
  source 1..22: mol_type = other DNA
tctcccaacccttgtaccagtg

SEQ ID NO: 13 - DNA, 23 bp, synthetic construct
  source 1..23: mol_type = other DNA
ttaatgctaatcgtgataggggt

SEQ ID NO: 14 - DNA, 22 bp, synthetic construct
  source 1..22: mol_type = other DNA
tagcagcacgtaaatattggcg

SEQ ID NO: 15 - DNA, 22 bp, synthetic construct
  source 1..22: mol_type = other DNA
catcttaccggacagtgctgga

SEQ ID NO: 16 - DNA, 22 bp, synthetic construct
  source 1..22: mol_type = other DNA
catcttactgggcagcattgga

SEQ ID NO: 17 - DNA, 23 bp, synthetic construct
  source 1..23: mol_type = other DNA
taatactgccgggtaatgatgga

SEQ ID NO: 18 - DNA, 22 bp, synthetic construct
  source 1..22: mol_type = other DNA
ttccctttgtcatcctatgcct

SEQ ID NO: 19 - DNA, 22 bp, synthetic construct
  source 1..22: mol_type = other DNA
tggaatgtaaggaagtgtgtgg

SEQ ID NO: 20 - DNA, 22 bp, synthetic construct
  source 1..22: mol_type = other DNA
tagcttatcagactgatgttga

SEQ ID NO: 21 - DNA, 22 bp, synthetic construct
  source 1..22: mol_type = other DNA
acagcaggcacagacaggcagt

SEQ ID NO: 22 - DNA, 24 bp, synthetic construct
  source 1..24: mol_type = other DNA
tactgcatcaggaactgattggat

SEQ ID NO: 23 - DNA, 22 bp, synthetic construct
  source 1..22: mol_type = other DNA
tagcttatcagactgatgttga

SEQ ID NO: 24 - DNA, 21 bp, synthetic construct
  source 1..21: mol_type = other DNA
tgtcagtttgtcaaatacccc

SEQ ID NO: 25 - DNA, 22 bp, synthetic construct
  source 1..22: mol_type = other DNA
cattgcacttgtctcggtctga

SEQ ID NO: 26 - DNA, 22 bp, synthetic construct
  source 1..22: mol_type = other DNA
ttcaagtaatccaggataggct

SEQ ID NO: 27 - DNA, 20 bp, synthetic construct
  source 1..20: mol_type = other DNA
ttcacagtggctaagttccg

SEQ ID NO: 28 - DNA, 22 bp, synthetic construct
  source 1..22: mol_type = other DNA
tagcaccatctgaaatcggtta

SEQ ID NO: 29 - DNA, 23 bp, synthetic construct
  source 1..23: mol_type = other DNA
tagcaccatttgaaatcagtgtt

SEQ ID NO: 30 - DNA, 22 bp, synthetic construct
  source 1..22: mol_type = other DNA
tagcaccatttgaaatcggtta

SEQ ID NO: 31 - DNA, 23 bp, synthetic construct
  source 1..23: mol_type = other DNA
tgtaaacatcctcgactggaagc

SEQ ID NO: 32 - DNA, 22 bp, synthetic construct
  source 1..22: mol_type = other DNA
tgtaaacatcctacactcagct

SEQ ID NO: 33 - DNA, 22 bp, synthetic construct
  source 1..22: mol_type = other DNA
tgtaaacatcctacactcagct

SEQ ID NO: 34 - DNA, 24 bp, synthetic construct
  source 1..24: mol_type = other DNA
tgtaaacatccccgactggaagct

SEQ ID NO: 35 - DNA, 22 bp, synthetic construct
  source 1..22: mol_type = other DNA
ctttcagtcggatgtttacagc

SEQ ID NO: 36 - DNA, 22 bp, synthetic construct
  source 1..22: mol_type = other DNA
tggcagtgtcttagctggttgt

SEQ ID NO: 37 - DNA, 22 bp, synthetic construct
  source 1..22: mol_type = other DNA
aatcattcacggacaacacttt

SEQ ID NO: 38 - DNA, 22 bp, synthetic construct
  source 1..22: mol_type = other DNA
aaaccgttaccattactgagtt

SEQ ID NO: 39 - DNA, 21 bp, synthetic construct
  source 1..21: mol_type = other DNA
tcctgtactgagctgccccga

SEQ ID NO: 40 - DNA, 23 bp, synthetic construct
  source 1..23: mol_type = other DNA
cagtaacaaagattcatccttgt

SEQ ID NO: 41 - DNA, 22 bp, synthetic construct
  source 1..22: mol_type = other DNA
tagcagcacgtaaatattggcg

SEQ ID NO: 42 - DNA, 22 bp, synthetic construct
  source 1..22: mol_type = other DNA
ctcgcttcggcagcacatatac

SEQ ID NO: 43 - DNA, 22 bp, synthetic construct
  source 1..22: mol_type = other DNA
tcaccgggtgtaaatcagcttg

SEQ ID NO: 44 - DNA, 24 bp, synthetic construct
  source 1..24: mol_type = other DNA
cgcaaggatgacacgcaaattcgt

SEQ ID NO: 45 - DNA, 21 bp, synthetic construct
  source 1..21: mol_type = other DNA
tcgaggagctcacagtctagt

SEQ ID NO: 46 - skipped

SEQ ID NO: 47 - DNA, 22 bp, synthetic construct
  source 1..22: mol_type = other DNA
caaagaattctccttttgggct

SEQ ID NO: 48 - DNA, 22 bp, synthetic construct
  source 1..22: mol_type = other DNA
ttcaagtaattcaggataggtt

SEQ ID NO: 49 - DNA, 22 bp, synthetic construct
  source 1..22: mol_type = other DNA
cactagattgtgagctcctgga

SEQ ID NO: 50 - RNA, 22 nt, synthetic construct
  source 1..22: mol_type = other RNA
tgaggtagtaggttgtgtggtt

SEQ ID NO: 51 - RNA, 22 nt, synthetic construct
  source 1..22: mol_type = other RNA
taccctgtagatccgaatttgt

SEQ ID NO: 52 - RNA, 22 nt, synthetic construct
  source 1..22: mol_type = other RNA
ccctgtagaaccgaatttgtgt

SEQ ID NO: 53 - RNA, 21 nt, synthetic construct
  source 1..21: mol_type = other RNA
cattattacttttggtacgcg

SEQ ID NO: 54 - RNA, 22 nt, synthetic construct
  source 1..22: mol_type = other RNA
cagtgcaatgttaaaagggcat

SEQ ID NO: 55 - RNA, 23 nt, synthetic construct
  source 1..23: mol_type = other RNA
taacagtctacagccatggtcgc

SEQ ID NO: 56 - RNA, 23 nt, synthetic construct
  source 1..23: mol_type = other RNA
tatggctttttattcctatgtga

SEQ ID NO: 57 - RNA, 23 nt, synthetic construct
  source 1..23: mol_type = other RNA
tatggcttttcattcctatgtga

SEQ ID NO: 58 - RNA, 21 nt, synthetic construct
  source 1..21: mol_type = other RNA
tgagatgaagcactgtagctc

SEQ ID NO: 59 - RNA, 22 nt, synthetic construct
  source 1..22: mol_type = other RNA
tacagtatagatgatgtactag

SEQ ID NO: 60 - RNA, 22 nt, synthetic construct
  source 1..22: mol_type = other RNA
tgagaactgaattccataggct

SEQ ID NO: 61 - RNA, 22 nt, synthetic construct
  source 1..22: mol_type = other RNA
tctcccaacccttgtaccagtg

SEQ ID NO: 62 - RNA, 23 nt, synthetic construct
  source 1..23: mol_type = other RNA
ttaatgctaatcgtgataggggt

SEQ ID NO: 63 - RNA, 22 nt, synthetic construct
  source 1..22: mol_type = other RNA
tagcagcacgtaaatattggcg

SEQ ID NO: 64 - RNA, 22 nt, synthetic construct
  source 1..22: mol_type = other RNA
catcttaccggacagtgctgga

SEQ ID NO: 65 - RNA, 22 nt, synthetic construct
  source 1..22: mol_type = other RNA
catcttactgggcagcattgga

SEQ ID NO: 66 - RNA, 23 nt, synthetic construct
  source 1..23: mol_type = other RNA
taatactgccgggtaatgatgga

SEQ ID NO: 67 - RNA, 22 nt, synthetic construct
  source 1..22: mol_type = other RNA
ttccctttgtcatcctatgcct

SEQ ID NO: 68 - RNA, 22 nt, synthetic construct
  source 1..22: mol_type = other RNA
tggaatgtaaggaagtgtgtgg

SEQ ID NO: 69 - RNA, 22 nt, synthetic construct
  source 1..22: mol_type = other RNA
tagcttatcagactgatgttga

SEQ ID NO: 70 - RNA, 22 nt, synthetic construct
  source 1..22: mol_type = other RNA
acagcaggcacagacaggcagt

SEQ ID NO: 71 - RNA, 24 nt, synthetic construct
  source 1..24: mol_type = other RNA
tactgcatcaggaactgattggat

SEQ ID NO: 72 - RNA, 22 nt, synthetic construct
  source 1..22: mol_type = other RNA
tagcttatcagactgatgttga

SEQ ID NO: 73 - RNA, 21 nt, synthetic construct
  source 1..21: mol_type = other RNA
tgtcagtttgtcaaatacccc

SEQ ID NO: 74 - RNA, 22 nt, synthetic construct
  source 1..22: mol_type = other RNA
cattgcacttgtctcggtctga

SEQ ID NO: 75 - RNA, 22 nt, synthetic construct
  source 1..22: mol_type = other RNA
ttcaagtaatccaggataggct

SEQ ID NO: 76 - RNA, 20 nt, synthetic construct
  source 1..20: mol_type = other RNA
ttcacagtggctaagttccg

SEQ ID NO: 77 - RNA, 22 nt, synthetic construct
  source 1..22: mol_type = other RNA
tagcaccatctgaaatcggtta

SEQ ID NO: 78 - RNA, 23 nt, synthetic construct
  source 1..23: mol_type = other RNA
tagcaccatttgaaatcagtgtt

SEQ ID NO: 79 - RNA, 22 nt, synthetic construct
  source 1..22: mol_type = other RNA
tagcaccatttgaaatcggtta

SEQ ID NO: 80 - RNA, 23 nt, synthetic construct
  source 1..23: mol_type = other RNA
tgtaaacatcctcgactggaagc

SEQ ID NO: 81 - RNA, 22 nt, synthetic construct
  source 1..22: mol_type = other RNA
tgtaaacatcctacactcagct

SEQ ID NO: 82 - RNA, 22 nt, synthetic construct
  source 1..22: mol_type = other RNA
tgtaaacatcctacactcagct

SEQ ID NO: 83 - RNA, 24 nt, synthetic construct
  source 1..24: mol_type = other RNA
tgtaaacatccccgactggaagct

SEQ ID NO: 84 - RNA, 22 nt, synthetic construct
  source 1..22: mol_type = other RNA
ctttcagtcggatgtttacagc

SEQ ID NO: 85 - RNA, 22 nt, synthetic construct
  source 1..22: mol_type = other RNA
tggcagtgtcttagctggttgt

SEQ ID NO: 86 - RNA, 22 nt, synthetic construct
  source 1..22: mol_type = other RNA
aatcattcacggacaacacttt

SEQ ID NO: 87 - RNA, 22 nt, synthetic construct
  source 1..22: mol_type = other RNA
aaaccgttaccattactgagtt

SEQ ID NO: 88 - RNA, 21 nt, synthetic construct
  source 1..21: mol_type = other RNA
tcctgtactgagctgccccga

SEQ ID NO: 89 - RNA, 23 nt, synthetic construct
  source 1..23: mol_type = other RNA
cagtaacaaagattcatccttgt

SEQ ID NO: 90 - RNA, 22 nt, synthetic construct
  source 1..22: mol_type = other RNA
tagcagcacgtaaatattggcg

SEQ ID NO: 91 - RNA, 22 nt, synthetic construct
  source 1..22: mol_type = other RNA
ctcgcttcggcagcacatatac

SEQ ID NO: 92 - RNA, 22 nt, synthetic construct
  source 1..22: mol_type = other RNA
tcaccgggtgtaaatcagcttg

SEQ ID NO: 93 - RNA, 24 nt, synthetic construct
  source 1..24: mol_type = other RNA
cgcaaggatgacacgcaaattcgt

SEQ ID NO: 94 - RNA, 21 nt, synthetic construct
  source 1..21: mol_type = other RNA
tcgaggagctcacagtctagt

SEQ ID NO: 95 - RNA, 22 nt, synthetic construct
  source 1..22: mol_type = other RNA
caaagaattctccttttgggct

SEQ ID NO: 96 - RNA, 22 nt, synthetic construct
  source 1..22: mol_type = other RNA
ttcaagtaattcaggataggtt

SEQ ID NO: 97 - RNA, 22 nt, synthetic construct
  source 1..22: mol_type = other RNA
cactagattgtgagctcctgga